(19) United States
(12) Patent Application Publication
(10) Pub. No.: US 2023/0123309 A1
(43) Pub. Date: Apr. 20, 2023

(54) ADAPTIVE PATIENT MONITORING SYSTEM FOR BED EXIT

(71) Applicant: Hill-Rom Services, Inc., Batesville, IN (US)

(72) Inventors: Timothy Receveur, Apex, NC (US); Eugene G. Urrutia, Apex, NC (US); Chiew Yuan Chung, Singapore (SG); Rachel L. Williamson, Batesville, IN (US); Sinan Batman, Cary, NC (US)

(21) Appl. No.: 17/964,996

(22) Filed: Oct. 13, 2022

Related U.S. Application Data
(60) Provisional application No. 63/255,604, filed on Oct. 14, 2021.

(51) Int. Cl. A61B 5/11; A61B 5/00

(52) U.S. Cl. CPC A61B 5/1117; A61B 5/746; A61B 5/1128

(57) ABSTRACT

A monitoring system for a patient fall risk protocol includes a support apparatus that includes a sensor configured to sense a position of a patient. A control unit is in communication with the sensor. The control unit is configured to monitor the position of the patient relative to a monitoring area on the support apparatus. The control unit is configured to determine a preliminary risk evaluation. A controller is in communication with the support apparatus. The controller is configured to receive the preliminary risk evaluation from the support apparatus and adjust a monitoring level of the monitoring area in response to the preliminary risk evaluation.

### CROSS-REFERENCE TO RELATED APPLICATION

This application claims priority to and the benefit under 35 U.S.C. § 119(e) of U.S. Provisional Application No. 63/255,604, filed on Oct. 14, 2021, entitled “ADAPTIVE PATIENT MONITORING SYSTEM FOR BED EXIT,” the disclosure of which is hereby incorporated herein by reference in its entirety.

### FIELD OF THE DISCLOSURE

The present disclosure generally relates to a patient monitoring system, and more particularly to an adaptive patient monitoring system for bed exit.

### SUMMARY OF THE DISCLOSURE

According to at least one aspect of the present disclosure, a monitoring system for a patient fall risk protocol includes a support apparatus that includes a sensor configured to sense a position of a patient. A control unit is in communication with the sensor. The control unit is configured to monitor the position of the patient relative to a monitoring area on the support apparatus. The control unit is configured to determine a preliminary fall risk evaluation. A controller is in communication with the support apparatus. The controller is configured to receive the preliminary fall risk evaluation from the support apparatus, determine an overall fall risk evaluation using the preliminary fall risk evaluation, assign a monitoring level for the monitoring area based on the overall fall risk evaluation, adjust the overall fall risk evaluation, and adjust the monitoring level of the monitoring area in response to changes in the overall fall risk evaluation.

According to another aspect of the present disclosure, a fall risk monitoring system for a medical facility includes a support apparatus including a sensor configured to sense position information related to a patient on the support apparatus and a control unit configured to monitor sensed patient information from the sensor relative to a monitoring trigger. A locator system is configured to determine a location of a caregiver in said medical facility. A controller is in communication with the support apparatus and the locator system. The controller is configured to assign a monitoring level of the monitoring trigger based on a fall risk evaluation, receive data from the support apparatus related to the patient and the support apparatus, receive the location of the caregiver, and adjust the monitoring level of the monitoring trigger in response to at least one of the data from the support apparatus and the location of the caregiver.

According to still another aspect of the present disclosure, an adaptive patient monitoring system includes a support apparatus having sensors, a servicer storing an electronic medical record, and a controller in communication with the support apparatus and the server. The controller is configured to receive information from the support apparatus relating to at least one of the support apparatus and a person on the support apparatus, receive data from the electronic medical record for the person, determine a monitoring level of a monitoring area on the support apparatus, and adjust the monitoring level in response to at least one of the information from the support apparatus and data from the electronic medical record.

According to various aspects of the present disclosure, a monitoring system provides an adaptive approach to adjusting a sensitivity of a fall risk protocol. Under a fall risk protocol, a patient may not exit a support apparatus without assistance. The adaptive monitoring system weighs various factors to determine an overall fall risk evaluation, which adjusts the sensitivity or monitoring level of the fall risk protocol. The various factors may be received from a call system, a coding system, a caregiver locator system, an electronic medical record, a support apparatus, a local server, a remote user device, additional inputs, or combinations thereof. The monitoring system is configured to continuously and dynamically adjust the monitoring level for the fall risk protocol to provide sufficient time for a caregiver to reach the patient attempting to exit a support apparatus. Further, the caregiver may provide feedback to the monitoring system to optimize the algorithms or routines that determine the sensitivity of the fall risk protocol, improving accuracy in the adaptive sensitivity determinations.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a side perspective view of a support apparatus in a patient room, according to the present disclosure;

FIG. 2 is a side perspective view of a support apparatus with an elevated head end and a lowered foot end, according to the present disclosure;

FIG. 3 is a block diagram of functionality associated with a support apparatus, according to the present disclosure;

FIG. 4 is a top plan view of a support apparatus having multiple monitoring areas illustrated on a support surface, according to the present disclosure;

FIG. 5 is a schematic diagram of a patient room with an imaging system having a field of view that includes a support apparatus, according to the present disclosure;

FIG. 6 is a block diagram of an adaptive monitoring system in communication with facility systems and devices, according to the present disclosure;

FIG. 7 is a block diagram is a support apparatus in wireless communication with a local servicer via a wireless access transceiver, according to the present disclosure;

FIG. 8 is a block diagram of a support apparatus wirelessly communicating with a local servicer via wireless access points, according to the present disclosure;

FIG. 9 is a side perspective view of a nurse station in a medical facility positioned outside patient rooms, according to the present disclosure;

FIG. 10 is a block diagram of a caregiver call system, according to the present disclosure;

FIG. 11 is a block diagram of a caregiver locator system, according to the present disclosure;

FIG. 12 is a block diagram of a monitoring system receiving various inputs to a fall risk protocol to adjust a sensitivity of the fall risk protocol and, consequently, a patient monitoring area, according to the present disclosure;

FIG. 13 is illustrative of an application interface having a feedback display for caregivers to input feedback for a fall risk protocol, according to the present disclosure;

FIG. 14 is illustrative of an application interface having a bed adjustment display for a caregiver to adjust bed features and activate an adaptive monitoring system, according to the present disclosure;

FIG. 15 is illustrative of an application interface displaying a fall risk alert notification, according to the present disclosure;

FIG. 16 is a flow diagram of a method for monitoring a patient under a fall risk protocol, according to the present disclosure; and

FIG. 17 is a flow diagram of a method for adjusting a fall risk protocol of a monitoring system, according to the present disclosure.

### DETAILED DESCRIPTION

The present illustrated embodiments reside primarily in combinations of method steps and apparatus components related to an adaptive patient monitoring system for bed exit. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

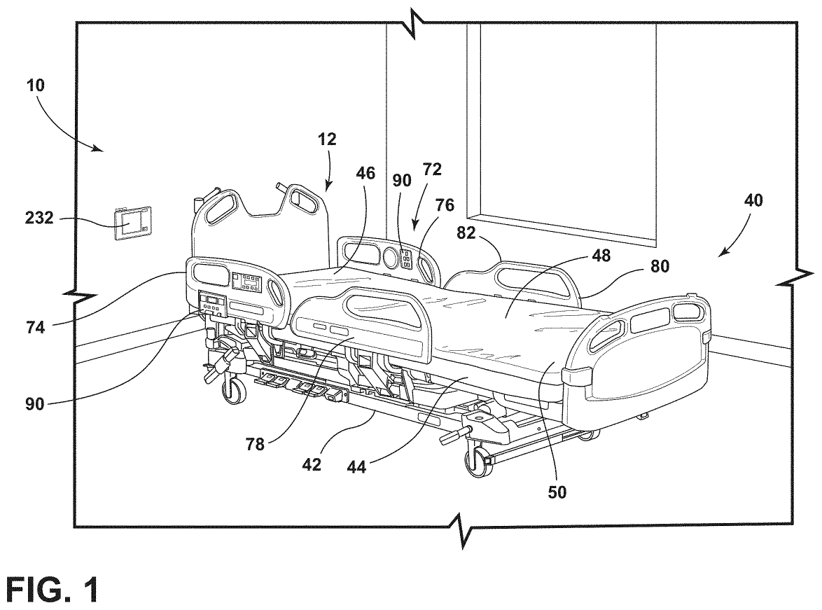

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

With reference to FIGS. 1-17, reference numeral 10 generally designates a monitoring system for a patient fall risk protocol that includes a support apparatus 12 having a sensor 14 configured to sense a position of a person or patient and a control unit 16 in communication with the patient position sensor 14. The control unit 16 is configured to monitor the position of the patient relative to a monitoring area 18 on the support apparatus 12. The control unit 16 is configured to determine a preliminary fall risk evaluation. A controller 20 is in communication with the support apparatus 12. The controller 20 is configured to receive the preliminary risk evaluation from the support apparatus 12, determine an overall fall risk evaluation using the preliminary fall risk evaluation, assign a monitoring level 22 for the monitoring area 18 based on the overall fall risk evaluation, adjust the overall fall risk evaluation, and adjust the monitoring level 22, such as by adjusting a boundary 24 of the monitoring area 18, in response to changes in the overall fall risk evaluation.

With reference to FIGS. 1-3, in the illustrated examples, the support apparatus 12 is configured as a bed positioned within a patient room 40. Though illustrated as the bed, the support apparatus 12 may be configured as a surgical table, stretcher, chair, or other structure for supporting a patient thereon. When configured as the bed, the support apparatus 12 generally includes a base frame 42 and an upper frame 44 coupled to the base frame 42. The upper frame 44 is adjustable relative to the base frame 42 (e.g., raise, lower, tilt, etc.). The upper frame 44 generally includes multiple segments 46, 48, 50 (e.g., the head end 46, the base region 48, and the foot end 50) that are independently adjustable relative to one another. The adjustable segments 46, 48, 50 allow the upper frame 44 to articulate between various positions, such as the elevated head end 46 and the lowered foot end 50.

The support apparatus 12 generally includes actuation assemblies 60 configured to adjust the upper frame 44. At least one actuation assembly 60 is configured to adjust the position of the upper frame 44 relative to the base frame 42 and, consequently, an underlying floor surface (e.g., a height of the support apparatus 12). Additionally, at least one actuation assembly 60 is configured to adjust the segments 46, 48, 50 of the upper frame 44 relative to one another. The actuation assemblies 60 may each include a motor 62 in communication with the control unit 16. The actuation assemblies 60 may have any practicable configuration without departing from the teachings herein.

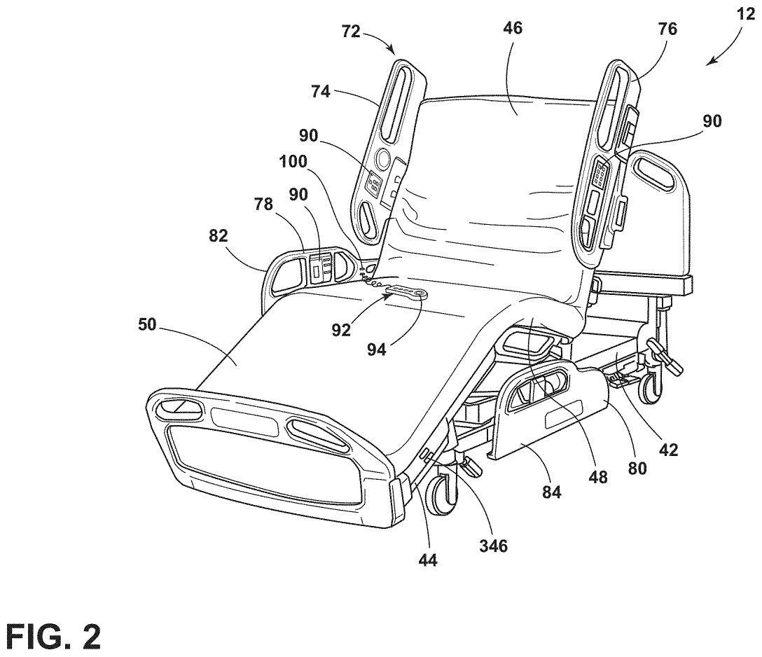

Referring still to FIGS. 1-3, the support apparatus 12 may include at least one bed sensor 70 configured to sense a position of the support apparatus 12. In certain aspects, the bed sensors 70 are configured to sense the position of the various segments 46, 48, 50 of the upper frame 44, including the head end 46, the foot end 50, and the base region 48. The bed sensors 70 may additionally or alternatively be configured to sense the height of the support apparatus 12 relative to the floor. In the example illustrated in FIG. 2, the bed sensor 70 would sense the angle of the head end 46 and the foot end 50 relative to a flat or base position, as well as the height of the base region 48 relative to the floor. The bed position information is communicated to the control unit 16, and the control unit 16 is configured to monitor the current position of the support apparatus 12, as well as trends, patterns, timing, etc. of the positions of the support apparatus 12. The position of the support apparatus 12 may be indicative of a potential bed exit attempt by the patient.

The support apparatus 12 includes multiple siderails 72, which are configured to be raised and lowered to selectively prevent or allow ingress and ingress to support apparatus 12. In the illustrated example, the support apparatus 12 includes two head siderails 74, 76 and two base siderails 78, 80, which are selectively and independently adjustable and collectively referred to herein as the siderails 72. The siderails 72 may be manually or automatically adjusted between raised and lowered positions 82, 84.

Referring still to FIGS. 1-3, the support apparatus 12 includes siderail sensors 86 configured to sense a position of the siderails 72. The siderail sensors 86 are configured to sense whether the siderails 72 are in the raised position 82 or the lowered position 84 and communicate the information to the control unit 16. The control unit 16 may monitor the current position of the siderails 72, as well as trends, patterns, timing, etc. for the adjustment of the siderails 72. The position of the siderails 72 in the lowered position 84 may be indicative of a potential bed exit attempt by the patient.

In the illustrated example of FIGS. 1-3, the support apparatus 12 includes siderail user interfaces 90 and a handheld user interface 92 coupled to the support apparatus 12. As illustrated, the support apparatus 12 includes multiple siderail user interfaces 90 to provide access to the patient and a caregiver based on the location or orientation of the selected siderail user interface 90. For example, the siderail user interfaces 90 on an inside of the siderails 72 may be for the patient to use, and the siderail user interfaces 90 on an outside of the siderails 72 may be for the caregiver to use.

The handheld user interface 92 may generally be referred to as a pillow speaker, which may be selectively coupled to the support apparatus 12. The handheld user interface 92 may control various aspects of the support apparatus 12 (e.g., raise, lower, etc.), features within the patient room 40 (e.g., television, room lighting, etc.), etc. In certain aspects, the handheld user interface 92 includes a call feature 94, also referred to as a nurse call feature 94, and may include a microphone 96 and a speaker 98 for communicating with the caregiver through the call feature 94. Each of the siderail user interfaces 90 and the handheld user interface 92 may be touchscreens, have touch-activated features, buttons, knobs, or have other selectable features. The selectable features may relate to the support apparatus 12, therapies provided to the patient, the call feature 94, or a combination thereof.

Referring still to FIG. 2, the support apparatus 12 may include one or more connector ports 100. The connector ports 100 may be, for example, USB ports, allowing the user to connect a personal device to the support apparatus 12. The control unit 16 is configured to monitor if a device is coupled with the connector port 100 and when the device is disconnected, which may be indicative of an attempt to exit the support apparatus 12 (e.g., a bed exit).

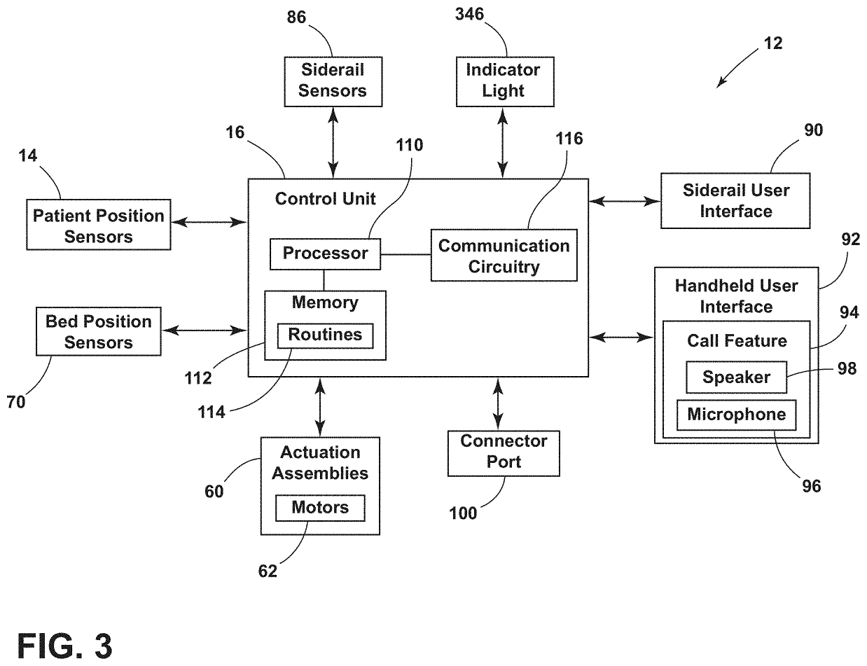

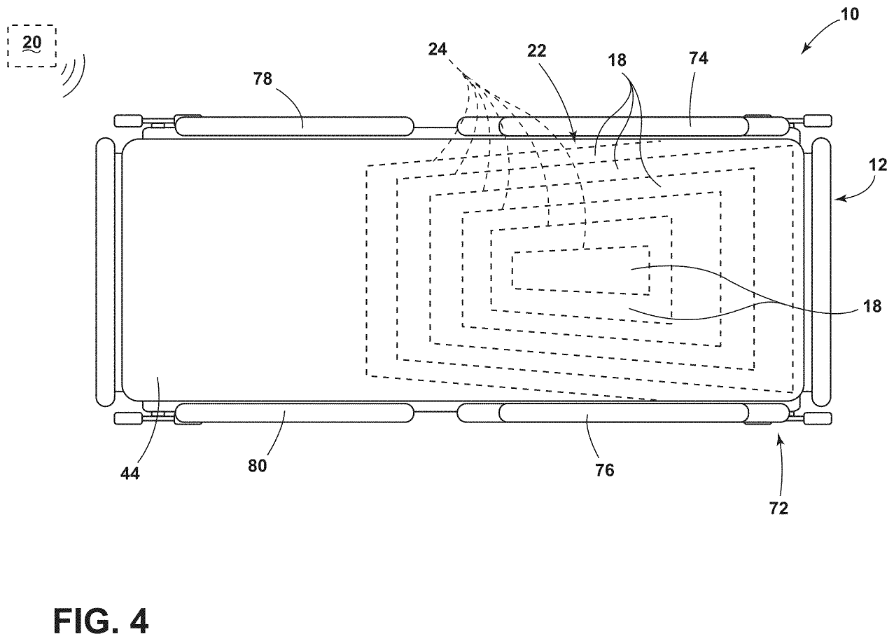

Referring now to FIG. 3 and FIG. 4, the support apparatus 12 includes at least one patient position sensor 14. Generally, the support apparatus 12 includes multiple patient position sensors 14 disposed along a support surface of the upper frame 44 of the support apparatus 12. The patient position sensors 14 may be load beams, load cells, pressure sensors, force sensors, weight sensors, or other similar sensors. The patient position sensors 14 are utilized to sense the position and movement of the patient on the support apparatus 12. In certain aspects, the patient position sensors 14 monitor a center of gravity of the patient. This position information of the patient is communicated to the control unit 16, which monitors current position information of the patient, as well as trends, patterns, and timing over a period of time.

The control unit 16 of the support apparatus 12 includes a processor 110, a memory 112, and other control circuitry. Instructions or routines 114 are stored with the memory 112 and executable by the processor 110. At least one of the routines 114 relates to determining the preliminary risk evaluation. Additionally, the control circuitry generally includes communication circuitry 116 configured for bidirectional wired or wireless communication.

The support apparatus 12 is included in or is in communication with the monitoring system 10 for the fall risk protocol. When the fall risk protocol is activated for the patient, the patient may not exit the support apparatus 12 unassisted (e.g., without the caregiver). Accordingly, monitoring the exit movements of the patient and alerting the caregiver of such movements is advantageous for preventing or minimizing fall hazards for the patient. The control unit 16 is configured to compile, weigh, analyze, and or otherwise utilize information from the support apparatus 12 to determine the preliminary risk evaluation for the patient attempting to exit the support apparatus 12 unassisted.

The preliminary risk evaluation is based on an algorithm or routine 114, which utilizes the information related to the support apparatus 12 and the patient to determine a risk level or risk status of the patient. The preliminary risk evaluation may be determined by the control unit 16 based on the information related to the support apparatus 12 and the patient without factoring in additional systems or other facility information. The risk level or risk status is generally any indication that the patient is at risk of exiting the bed unassisted and the likelihood the patient will exit the bed unassisted.

The preliminary risk evaluation may be a scale or value out of a predefined value, such as a value out of a total, a percentage, etc. Additionally or alternatively, the preliminary risk evaluation may be a risk category, such as low, medium, or high. In additional non-limiting examples, the preliminary risk evaluation may be designating the patient as “at risk” or “not at risk.” The control unit 16 is configured to compile and analyze various information about the support apparatus 12 and the patient thereon to determine the preliminary risk evaluation. The control unit 16 is configured to receive information from the patient position sensors 14, including patient position and movement, as well as the bed sensors 70 and the siderail sensors 86 related to the position of the support apparatus 12 and the siderails 72, respectively. Information from the support apparatus 12 that may be utilized by the control unit 16 to determine the preliminary risk evaluation includes, but is not limited to: a current bed position, current bed height relative to an underlying floor surface, bed articulation, status of brakes, a time since the last bed exit, button presses (e.g., for assistance, comfort, therapies, etc.), current patient position, patient activity or movement, patient leg position relative to an edge of the support apparatus 12, current siderail 72 positions, patient balance (e.g., trunk strength), a gait surrogate (e.g., time to move own mass), level of consciousness, sleep pattern, exit versus non-exit movements, mobility from a state of rest, agitation, acceleration of movements, multiple unaided bed exits, number of attempts to rise, time to rise, device disconnect from connector port 100, etc.

In various aspects, the control unit 16 is configured to monitor the sensed position information over time (e.g., initial and updated information). The position of the patient may be indicative of an exit movement, where the patient is attempting to exit the support apparatus 12. For example, the control unit 16 is configured to determine when the patient is moving in a manner indicative of bed egress or exit through the patient position sensors 14, such as legs moving toward an edge of the support apparatus 12, center of gravity of the patient moving relative to the boundary 24, etc. In another example, the control unit 16 may determine bed conditions are indicative of bed exit via the siderail sensors 86, such as lowered siderails 72, and the bed sensors 70, such as a lowered height of the support apparatus 12.

As illustrated in FIG. 4, the controller 20 of the monitoring system 10 is in communication with the control unit 16 of the support apparatus 12. The monitoring system 10 is configured to receive the preliminary risk evaluation determined by the control unit 16, as well as the information the control unit 16 utilized to determine the preliminary risk evaluation. The controller 20 defines the monitoring area 18 on the support apparatus 12, which may be based, at least in part, on the preliminary risk evaluation from the control unit 16 of the support apparatus 12.

The controller 20 is configured to determine an overall fall risk evaluation, which may be the risk level or risk status that indicates that the patient is at risk of exiting the bed unassisted and the likelihood the patient will exit the bed unassisted. The overall risk evaluation may be based on a variety of information from multiple information sources related to the support apparatus 12, the patient, the medical facility, etc. The overall risk evaluation is continually and dynamically updated in response to each new information received by the controller 20.

The controller 20 of the monitoring system 10 is configured to assign and adjust the monitoring level 22 for the patient under the bed exit protocol based on the overall risk evaluation and changes to the overall risk evaluation. The monitoring level 22 is configured to be dynamically updated based on changing information related to the patient, the support apparatus 12, the medical facility, etc. The monitoring system 10 is configured to use the monitoring level 22 to determine an attempted exit from the support apparatus 12 and alert the caregiver, where the timing of the alert is adjusted by the monitoring system 10 as described herein.

The monitoring system 10 is configured to determine whether an exit is being attempted by comparing sensed patient information with a monitoring trigger, such as predefined locations, areas, or thresholds, and alert the caregiver. For example, the monitoring system 10 may compare the patient position information with a select or predefined location or locations (e.g., an area within a predefined distance to one or both sides of the support apparatus 12), the patient position information with the boundary 24 on the support apparatus 12, patient movement above a movement threshold, or other practicable comparisons. The monitoring level 22 may be implemented as spatial sensitivity or movement sensitivity. Each of the distance to the predefined location, the movement threshold, and the boundary 24 may collectively be referred to as the monitoring trigger, which are adjustable to trigger when the caregiver is alerted of a bed exit attempt by the patient.

The monitoring level 22 may initially be assigned based on the preliminary risk evaluation and/or any initial or preliminary assessments of the patient when the monitoring system 10 is activated. In various aspects, the monitoring area 18 is defined within the boundary 24 set by the controller 20 of the monitoring system 10. The boundary 24 may be adjusted by the controller 20 in response to a variety of information, as described further herein. The boundary 24 may be adjusted to increase a size of the monitoring area 18, and consequently decrease a sensitivity of the fall risk protocol, or to decrease the size of the monitoring area 18, and consequently increase the sensitivity of the fall risk protocol.

In additional examples, the controller 20 may be configured to monitor a position of the patient relative to a predefined location or locations. The predefined location may be a side of a mattress or surface assembly on the support apparatus 12 or both sides of the mattress. In such examples, the monitoring level 22 may be based on an area within a predefined distance to the predefined location. The controller 20 may monitor the position of the patient relative to the area within the distance to the predefined location. The monitoring level 22 may be adjusted to adjust the distance from the predefined location that causes the caregiver to be alerted of an attempted bed exit. For example, a larger distance from the predefined location may be utilized to increase the sensitivity of the fall risk protocol, allowing a greater time for the caregiver to reach the patient, and a smaller distance from the predefined location may be utilized to decrease the sensitivity of the fall risk protocol. It is contemplated that the predefined location may be any location, such as sides of the mattress, a centerline of the mattress, etc.

In another non-limiting example, the controller 20 may be configured to monitor movement of the patient relative to a predefined movement threshold. The monitoring level 22 may be the amount of movement from the patient that triggers the caregiver being notified of the bed exit attempt. In such examples, the monitoring level 22 of the fall risk protocol may be adjusted to define the predefined movement threshold. The predefined movement threshold may be decreased to increase the sensitivity of the fall risk protocol. In this way, less movement by the patient is configured to reach the predefined movement threshold and alert the caregiver. The predefined movement threshold may be increased to decrease the sensitivity of the fall risk protocol.

The monitoring level 22 corresponds with the sensitivity of the fall risk protocol. The sensitivity of the fall risk protocol adjusts the timing for when the caregiver is alerted of a potential bed exit attempt relative to movement of the patient. For example, when the sensitivity is higher, an earlier movement in an exiting process, such as moving from supine to sitting, will result in the caregiver being alerted. On the other hand, when the sensitivity is lower, a later movement of the exiting process, such as legs moving toward or off the edge of the support apparatus 12, will result in the caregiver being alerted. The higher sensitivity results in an earlier alert compared to the lower sensitivity, allowing more time between the alert and the bed exit attempt by the patient.

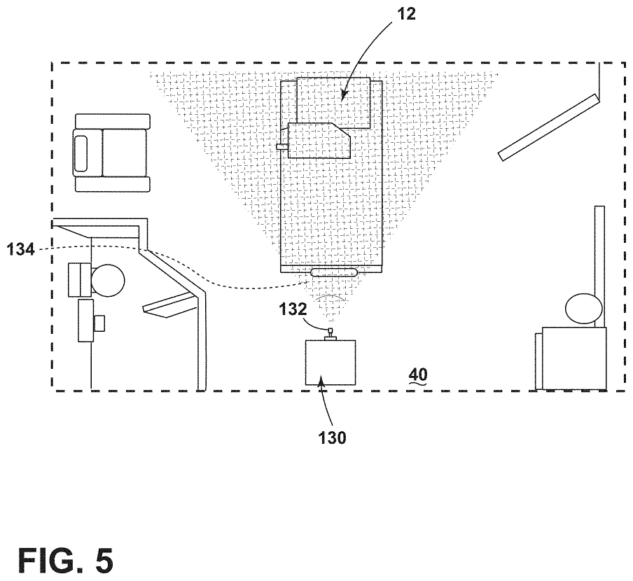

With reference to FIG. 5, in addition to data or information from the support apparatus 12, the monitoring system 10 may include or be in communication with an imaging system 130. The imaging system 130 is positioned within the patient room 40 and may be utilized to monitor the movement of the patient and a presence of the caregiver. The imaging system 130 generally includes an imager 132 defining a field of view 134 that encompasses at least the support apparatus 12. The imager 132 is configured to capture image data from within the field of view 134 and ultimately communicate the information to the monitoring system 10. The image data may be utilized by the monitoring system 10 to determine a variety of information about the patient, including the position on the bed, trunk strength, movement as a surrogate to gait (e.g., time to move own mass, such as moving from supine to sitting), and other information relevant to the fall risk protocol. In various aspects, the field of view 134 may extend to an area adjacent to the support apparatus 12 or other locations in the patient room 40. In such examples, the imaging system 130 may be utilized to monitor the gait of the patient as the patient ambulates about the patient room 40.

The imager 132 may have multiple configurations. The imager 132 may be or include a charge coupled device (CCD), complementary metal oxide semiconductor (CMOS), color camera, black-and-white camera, infrared camera, infrared image sensor, three-dimensional image sensor, or any other type of image sensor. The image data may include at least one of a picture, a video, real-time streaming of image data, other transmissions of image data, or combinations thereof. The imager 132 may be adjustable. In various examples, the imager 132 may be adjusted to change the scope of the field of view 134. It is contemplated that the imager 132 may include one or more lenses, which may be adjusted to change the sharpness or quality of the image data obtained by the imager 132. The field of view 134 may also be adjustable to be broader, narrower, positionally shifted, or any combination thereof.

The imaging system 130 includes a controller having a processor, a memory, and other control circuitry, including communication circuitry. Instructions or routines are stored within the memory and executable by the processor. At least one routine of the imaging system 130 may be directed to image processing. Additionally, at least one routine of the imaging system 130 may be utilized to adjust the imager 132 or the field of view 134.

The monitoring system 10 may also utilize the imaging system 130 to determine information about the caregiver and the interaction between the patient and the caregiver. For example, the monitoring system 10 is configured to analyze the image data to determine if and when the caregiver arrives at and is in the patient room 40. In such examples, the monitoring system 10 is configured to determine when the caregiver enters the patient room 40, which affects an alert generated by the monitoring system 10. Additionally or alternatively, the monitoring system 10 may determine whether the patient relies on the caregiver to rise from the support apparatus 12 or to ambulate about the patient room 40.

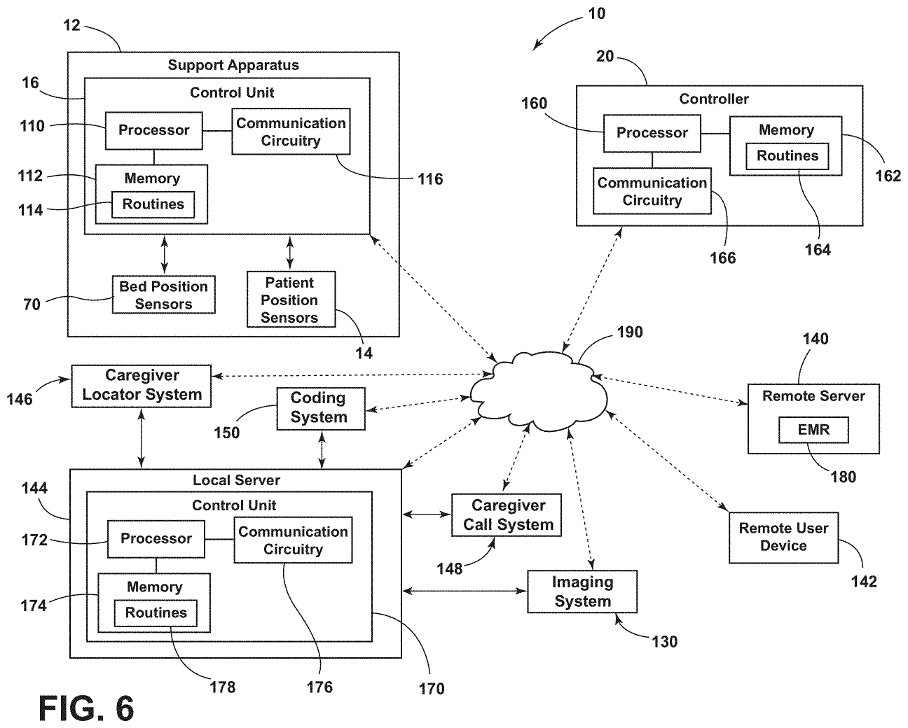

With reference to FIG. 6, the monitoring system 10 includes or is in communication with a variety of information sources to compile information and data that is utilized to adjust the sensitivity or monitoring level 22 of the fall risk protocol of the monitoring system 10. The information sources include, but are not limited to, the support apparatus 12, the imaging system 130, a remote server 140, a remote user device 142, a local server 144, a caregiver locator system 146, a call system 148 (e.g., a nurse call system 148), a coding system 150, etc.

The monitoring system 10 includes the controller 20 having a processor 160, a memory 162, and other control circuitry. Instructions or routines 164 are stored with the memory 162 and executable by the processor 160. At least one of the routines 164 may be directed to compiling, weighing, and/or analyzing information and data from the various information sources described herein. Additionally, at least one routine 164 may be directed to determining an overall risk evaluation of the patient and, consequently, adjusting the sensitivity of the fall risk protocol. The control circuitry includes communication circuitry 166 configured for bidirectional wired or wireless communication with the various information sources.

The controller 20 is in communication with the local server 144, which has a control unit 170 with a processor 172, a memory 174, and other control circuitry, including communication circuitry 176 for bidirectional wired and wireless communication. Instructions or routines 178 are stored in the memory 174 and executable by the processor 172. The local server 144 generally includes software (e.g., routines 178) relating to a variety of facility systems, including the monitoring system 10, the call system 148, the imaging system 130, the locator system 146, and the coding system 150. Information may be stored or communicated through the local server 144, which may be utilized by the monitoring system 10.

With reference still to FIG. 6, the controller 20 of the monitoring system 10 is also in communication with the remote server 140. The remote server 140 is configured for bidirectional wired or wireless communication. The remote server 140 generally stores electronic medical records (EMRs) 180 for the patients of the medical facility. The EMR 180 for each patient includes a variety of information (e.g., stored patient information) that may be utilized by the monitoring system 10, including, for example, bibliographic information, medication information (e.g., introduction, wear off, effect on coordination, etc.), medical conditions (e.g., seizures, etc.), history of falls, fall risk scores, other fall risk data, etc. This information may be retrieved by or communicated to the monitoring system 10 to be used in the overall fall risk evaluation determination.

The controller 20 is also in communication with the remote user device 142. The remote user device 142 may generally be a personal device of the caregiver, such as a phone, tablet, wearable device, laptop, or other remote communication devices. The remote user device 142 generally includes software or an application that allows the caregiver to input information about the patient into the remote user device 142, communicate with other caregivers, and view alerts. The monitoring system 10 may receive information about the patient from the remote user device 142, as well as alert the caregiver of the bed exit information (e.g., protocol activation, exit attempt, etc.) via the remote user device 142. It is contemplated that the controller 20 may be included at least partially in the local server 144, the remote server 140, the remote user device 142, or a combination thereof without departing from the teaching herein.

With reference still to FIG. 6, each of the controller 20 and the various information sources are configured to communicate wirelessly through a communication interface 190. The communication interface 190 may be part of a network of the medical facility. The network may include a combination of wired connections (e.g., Ethernet 192), as well as wireless connections, which may include the wireless communication interface 190. The communication interface 190 may include a variety of electronic devices and servers, which may be configured to communicate over various wired or wireless communication protocols. The communication interface 190 may include a wireless router through which the remotely accessed devices may be in communication with one another, as well as the local server 144

The communication interface 190 may be implemented via one or more direct or indirect nonhierarchical communication protocols, including but not limited to, Bluetooth®, Bluetooth® low energy (BLE), Thread, Ultra-Wideband, Z-wave, ZigBee, etc. Additionally, the communication interface 190 may correspond to a centralized or hierarchal communication interface 190 where one or more of the devices communicate via the wireless router (e.g., a communication routing controller). Accordingly, the communication interface 190 may be implemented by a variety of communication protocols, including, but not limited to, global system for mobile communication (GSM), general packet radio services, code division multiple access, enhanced data GSM environment, fourth generation (4G) wireless, fifth generation (5G) wireless, Wi-Fi, world interoperability for wired microwave access (WiMAX), local area network, Ethernet 192, etc. By flexibly implementing the communication interface 190, the various devices and servers may be in communication with one another directly via the wireless communication interface 190 or a cellular data connection.

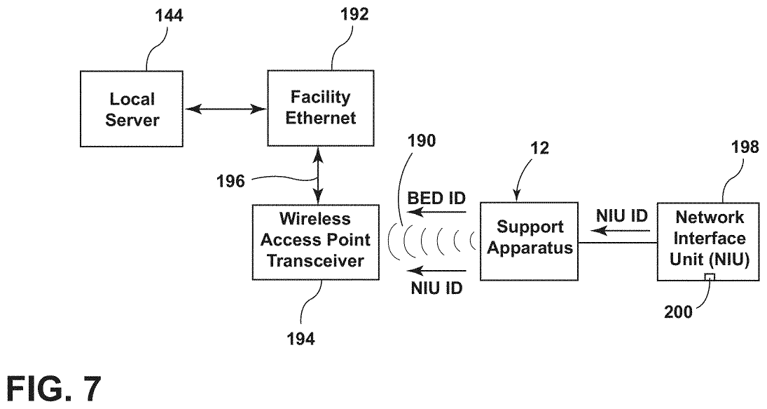

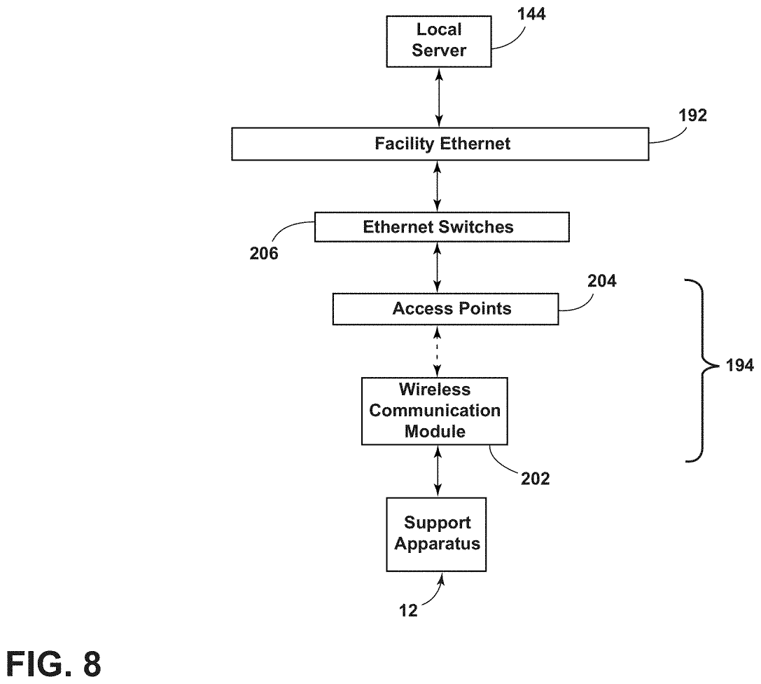

Referring still to FIG. 6, as well as FIGS. 7 and 8, exemplary communications of the support apparatus 12 to the local server 144 are illustrated. The monitoring system 10 may receive information from the support apparatus 12 via the communication interface 190 and through the local server 144. In certain aspects, the support apparatus 12 is configured to communicate with a wireless access transceiver 194, which is coupled to Ethernet 192 of the medical facility. The communication interface 190 provides for bidirectional communication between the support apparatus 12 and the wireless access transceiver 194. The wireless access transceiver 194 communicates bidirectionally with Ethernet 192 via a data link 196.

As illustrated in FIG. 7, the support apparatus 12 may be associated with a network interface unit 198. Multiple network interface units 198 may be provided in various locations within the medical facility. Each support apparatus 12 and each network interface unit 198 is assigned a unique identification code, such as a serial number. Various components of the monitoring system 10 on the local server 144 include software (e.g., routines 178) that operate to associate the identification code of the support apparatus 12 with the network interface unit 198 identification data to locate each support apparatus 12 within the medical facility. Each network interface unit 198 includes a port 200 for selectively coupling with Ethernet 192. When the network interface unit 198 is coupled with Ethernet 192, the network interface unit 198 communicates the identification data to the support apparatus 12, which then wirelessly communicates the data for the support apparatus 12 and the network interface unit 198 to the wireless access transceiver 194. The wireless access transceiver 194 then communicates with the local server 144 via Ethernet 192.

As illustrated in FIG. 8, the support apparatus 12 may be capable of communicating wirelessly via a wireless communication module 202. The wireless communication module 202 generally communicates via an SPI link with circuitry of the associated support apparatus 12 (e.g., the communication circuitry 116) and via a wireless 802.11 link with wireless access points 204. The wireless access points 204 are generally coupled to Ethernet switches 206 via 802.3 links. It is contemplated that the wireless communication modules 202 may communicate with the wireless access points 204 via any of the wireless protocols disclosed herein. Additionally or alternatively, the Ethernet switches 206 may generally communicate with Ethernet 192 via an 802.3 link. Ethernet 192 is also in communication with the local server 144, allowing information and data to be communicated between the local server 144 and the support apparatus 12.

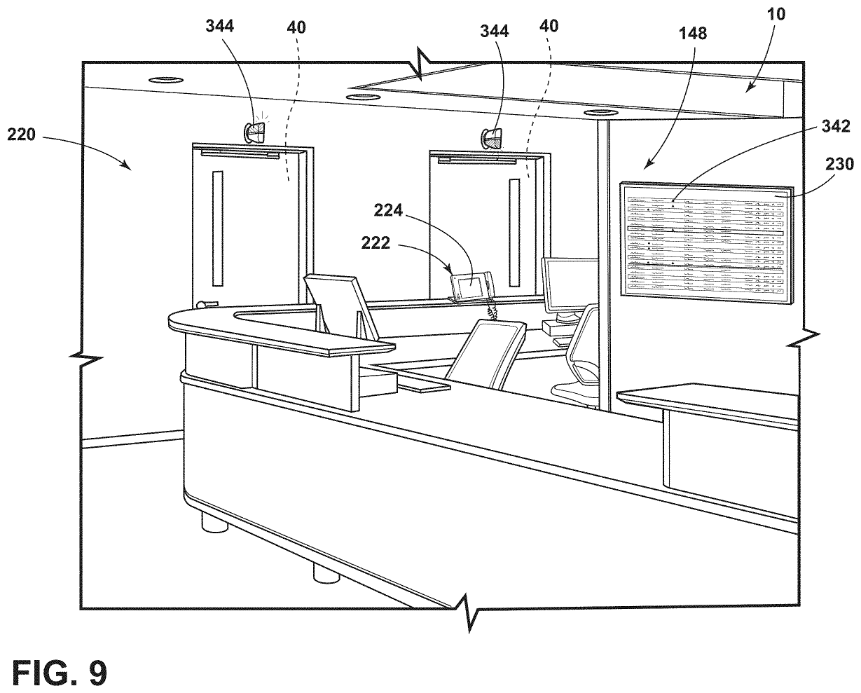

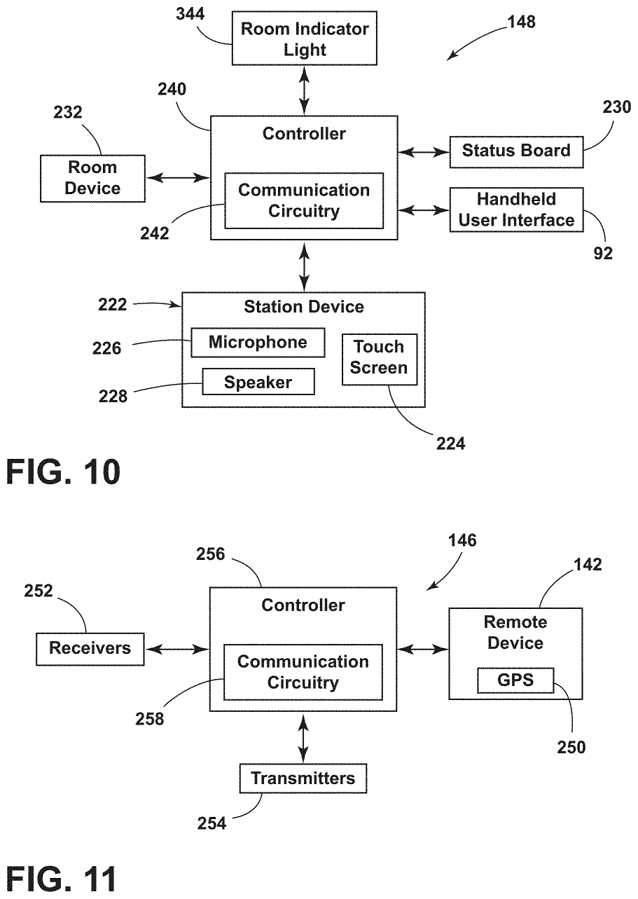

Referring again to FIG. 6, as well as to FIGS. 9 and 10, the monitoring system 10 includes or is in communication with the call system 148. The call system 148 provides communication between the patient and the caregiver, as well as other members of the caregiving team. The call system 148 generally connects the patient in the patient room 40 with the caregiver and with a nurse station 220. An exemplary nurse station 220 is illustrated in FIG. 9 and includes a station device 222, which is generally a device located at the nurse station 220 within the medical facility, such as a computer, a laptop, a tablet, etc. In the illustrated configuration, the station device 222 includes a touch screen 224, buttons, a microphone 226, and a speaker 228. Further, the call system 148 generally includes a status board 230 located at the nurse station 220 or elsewhere in the medical facility. The status board 230 includes information about various patients, including call information, fall risk status, etc. The call information may include that the patient is currently calling for the caregiver, a recent call time, etc.

The call system 148 may include a room device 232 coupled with a wall surface within the patient room 40 (as illustrated in FIG. 1) to receive inputs from the caregiver within the patient room 40, such as during rounds or when assisting with a bed exit. Further, the call system 148 may include the handheld user interface 92 coupled to the support apparatus 12 (as illustrated in FIG. 2) for the patient to call for the caregiver from the support apparatus 12. Moreover, the call system 148 is generally in communication with the remote user device 142 to provide call information from the patient directly to the caregiver, who may not be located at the nurse station 220.

The call system 148 includes a controller 240 having a processor, a memory, and other control circuitry. Instructions or routines are stored in the memory and executable by the processor. The control circuitry includes communication circuitry 242 for bidirectional wired or wireless communication. Each component of the call system 148 may communicate information to and receive information from the controller 240, which also communicates selected information to the monitoring system 10. Such information may include type of calls, quantity of calls, etc. The call system 148 is utilized for communicating information to the caregiver about the fall risk protocol and the patient, as well as for providing information to the monitoring system 10.

Referring still to FIG. 6, as well as to FIG. 11, the monitoring system 10 includes or is in communication with the caregiver locator system 146. The locator system 146 monitors the location of the caregiver within the medical facility, which may be communicated to the monitoring system 10 for adjusting the sensitivity of the fall risk protocol. The locator system 146 may include different configurations depending on the medical facility. In a non-limiting example, the locator system 146 is in communication with the remote user device 142 to obtain the location of the caregiver. In such examples, the application or software included in the remote user device 142 utilizes a global positioning system (GPS) 250 to determine location information or data of the user of the remote user device 142. The location information may be communicated to the monitoring system 10. Using GPS 250, the locator system 146 may provide real-time location information.

In another non-limiting example, the locator system 146 may utilize receivers 252 and transmitters 254 to determine the location of the caregiver. In such examples, the receivers 252 are positioned in various locations throughout the medical facility. The transmitters 254 are worn or carried by the caregivers, for example in identification (ID) badges or locator tags. Each ID badge may have a unique ID to be detected by the receivers 252. The ID detected by the receiver 252 is communicated to a controller 256 of the locator system 146. The controller 256 may determine who the caregiver is based on the unique ID and the location based on the receiver 252 that detected the ID. The locator system 146 may utilize one or both of the GPS 250 from the remote user device 142 and the transmitters 254 and receivers 252 without departing from the teachings herein.

The locator system 146 includes the controller 256, which has a processor, a memory, and other control circuitry. Instructions or routines are stored in the memory and executable by the processor. The control circuitry includes communication circuitry 258 for bidirectional wired or wireless communication. The caregiver ID and location are communicated from the locator system 146 to the monitoring system 10, which may be advantageous for adjusting a predicted time for the caregiver to reach a selected patient under the fall risk protocol.

The controller 20 of the monitoring system 10 may store estimated times to travel between select locations at the medical facility. In various aspects, the estimated times may be utilized to determine the predicted time for the caregiver to reach the specific patient room. The predicted time for the caregiver to reach the patient room at any given time may be utilized to adjust the monitoring level 22 of the monitoring system 10. The monitoring system 10 adapts for the location of the caregiver within the medical facility, rather than relying on predefined timing for alerting the caregiver.

Referring again to FIG. 6, the monitoring system 10 may also be in communication with a coding system 150 of the medical facility. Generally, the coding system 150 is an alert system that alerts the caregiving team of a medical alert or code for certain patients. The coding system 150 is configured to trigger the alert for the patient room, the unit, the floor, or the facility depending on the type of code. Additionally, the coding system 150 may be triggered by a caregiver input or may be triggered automatically. In automatic examples, the coding system 150 is in communication with various medical or treatment devices, such as vital sign monitors, and a change in the detected patient data may trigger the coding system 150. Whether a room, floor, unit, or area is under a code alert may affect the availability of the caregiving team for other patients, including those under the fall risk protocol. Additionally, when certain caregivers are responding to a code, the next available caregivers may be a further distance from patients under the fall risk protocol, as determined by the locator system 146. This facility code or coding information is communicated to the monitoring system 10 to allow the monitoring system 10 to adjust the sensitivity of the fall risk protocol in response to the availability of the caregiver and the location of additional caregivers.

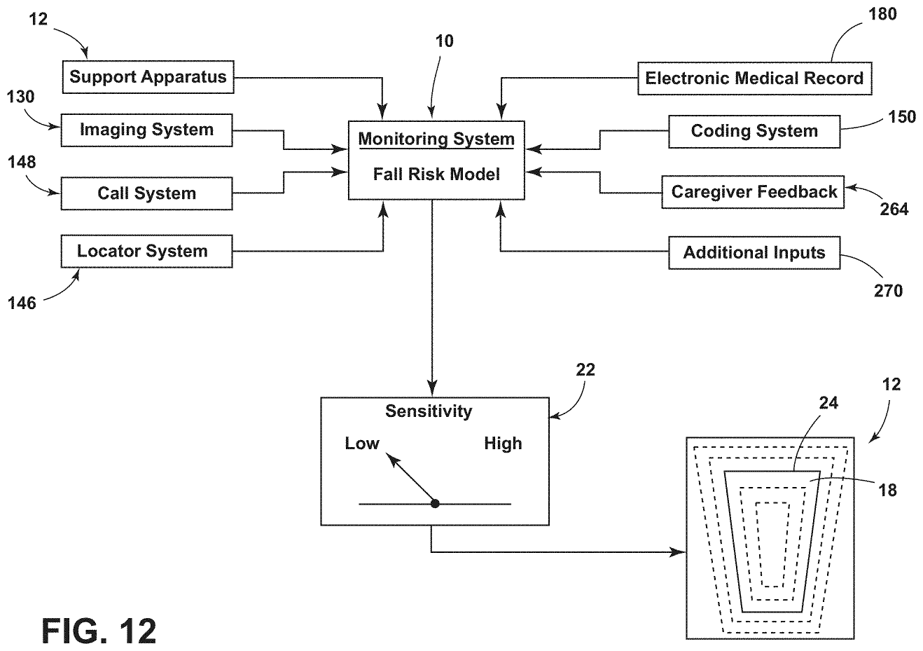

With reference to FIGS. 6 and 12, the monitoring system 10 is utilized for adjusting the sensitivity of the fall risk protocol. When the fall risk protocol is activated for the patient, the patient may not exit the bed without the assistance of the caregiver due to a heightened risk that the patient may fall or be injured. The monitoring system 10 is configured to alert the caregiver when the patient is attempting to exit the bed (e.g., the support apparatus 12) unassisted. The monitoring system 10 is adaptive based on the information received from at least one of the various information sources to minimize the risk that the patient will exit the support apparatus 12 and fall, as well as to reduce alarm fatigue for the caregiver by reducing the number of alarms throughout a shift.

The monitoring system 10 is configured to dynamically adjust the fall risk protocol based on a variety of factors, which the controller 20 is configured to compile, weigh, and analyze in accordance with one or more routines 164. At least one routine 164 of the monitoring system 10 is configured to receive, retrieve, or otherwise obtain information regarding the patient, the support apparatus 12, the caregiver, and other circumstances of the medical facility to adjust the sensitivity of the fall risk protocol. The information or factors utilized by the monitoring system 10 are generally obtained from one or more of the support apparatus 12, the imaging system 130, the call system 148, the locator system 146, the coding system 150, the EMR 180, the remote user device 142, caregiver feedback 264, additional inputs 270, etc., as described further herein.

The support apparatus 12 provides a variety of information regarding the support apparatus 12 (e.g., bed information) and the patient positioned thereon. This information may be provided directly and/or as-analyzed in the preliminary risk evaluation. The patient position sensors 14, the bed sensors 70, and the siderail sensors 86 each sense information and communicate the information to the control unit 16, which is subsequently communicated to the monitoring system 10. Information from the support apparatus 12 that may be utilized by the monitoring system 10 includes, but is not limited to: a current bed position, current bed height, bed articulation, status of brakes, a time since the last bed exit, button presses (e.g., for assistance, comfort, therapies, etc.), current patient position, patient activity or movement, patient leg position relative to an edge of the support apparatus 12, current siderail 72 positions, patient balance (e.g., trunk strength), a gait surrogate (e.g., time to move own mass), level of consciousness, sleep pattern, exit versus non-exit movements, mobility from a state of rest, agitation, acceleration of movements, multiple unaided bed exits, number of attempts to rise, time to rise, device disconnect from connector port 100, etc.

With reference still to FIGS. 6 and 12, the monitoring system 10 may analyze the information from the support apparatus 12 in accordance with one or more routines 164. For example, the monitoring system 10 may find a pattern in exit movements, where the patient intends to exit the support apparatus 12, and non-exit movements, where the patient does not intend to exit the support apparatus 12. Certain movements, times of day, and other factors may allow the monitoring system 10 to distinguish between exit and non-exit movements. In such examples, the patient moving his or her legs toward an edge of the support apparatus 12 proximate to one of the base siderails 78, 80 in the lowered position 84 may indicate an exit movement.

In another non-limiting example, the monitoring system 10 may determine a number of attempts the patient uses to rise from the support apparatus 12. An attempt may be detected by a lesser force or weight detected by the patient position sensors 14 followed by an increase in the sensed force or weight. The patient position sensors 14 may sense a lesser force or weight and then no or minimal force or weight when the patient has successfully risen from the support apparatus 12. The information from the support apparatus 12 may be received, analyzed, and weighted to contribute to the overall fall risk evaluation determined by the monitoring system 10.

In certain aspects, the control unit 16 of the support apparatus 12 may provide the preliminary fall risk evaluation based on the information from the support apparatus 12, which is communicated to the monitoring system 10. For example, the control unit 16 may monitor or analyze single events, patterns of the events over a predefined period of time, trends in the events, or a combination thereof. The control unit 16 may determine the preliminary risk evaluation for the fall risk protocol utilizing the information, the analysis of the information, or a combination thereof. The preliminary risk evaluation may be a determination of the risk severity of the patient based on the information detected or sensed by the support apparatus 12. The control unit 16 weighs, compiles, or otherwise utilizes the information received from the support apparatus 12 to determine the preliminary risk evaluation. The preliminary risk evaluation may be a calculated or determined score, number, or scale based on the information from the support apparatus 12, which affects the sensitivity of the fall risk protocol.

The support apparatus 12 may send the sensed information, the analysis of the information, the preliminary risk evaluation, or a combination thereof to the monitoring system 10. The monitoring system 10 may use any, some, or all of the information and data provided by the support apparatus 12 to determine the overall risk evaluation for the fall risk protocol.

Referring still to FIGS. 6 and 12, the monitoring system 10 may also utilize information from the imaging system 130 to adjust the sensitivity of the fall risk protocol. The imaging system 130 captures image data that includes the support apparatus 12. Depending on the configuration of the imaging system 130, the imagining system 130 may also capture image data for the area adjacent to the support apparatus 12 and/or other areas of the patient room 40. The imaging system 130 may provide information similar to the support apparatus 12, as well as additional information. The image data obtained by the imaging system 130 may be utilized by the controller of the imaging system 130 and/or the controller 20 of the monitoring system 10 to determine information that may affect the sensitivity of the fall risk protocol. One or both of the imaging system 130 and the monitoring system 10 may analyze the captured image data to obtain information.

The information and data from the image data that may be used by the monitoring system 10 include, but are not limited to: trunk strength (e.g., to replace gait when the patient is on the support apparatus 12), movement of the patient on the support apparatus 12, gait and ambulation in the patient room 40 (e.g., when the patient is out of the support apparatus 12), movement of the support apparatus 12, whether the caregiver is in the patient room 40, caregiver proximity to the patient, balance of the patient in the support apparatus 12, balance of the patient out of the support apparatus 12, use of assistive devices for moving off the support apparatus 12 to a standing position, use assistive devices for walking/ambulating, movement or ambulation to the caregiver or the assistive device, etc. The monitoring system 10 may monitor single events, patterns of the events over a predefined period of time, trends in the events, or a combination thereof. The information from the imaging system 130 may be utilized to adjust the sensitivity of the fall risk protocol.

Referring still to FIGS. 6 and 12, other systems in the medical facility may also provide information to the monitoring system 10 to be factored into the overall risk evaluation, which is utilized to adjust the sensitivity of the fall risk protocol. These additional systems may provide patient information, caregiver information, or facility information. For example, the call system 148 may communicate a variety of information to the monitoring system 10. This information from the call system 148 may include, but is not limited to: bed data (e.g., position, therapies, attachments, etc.), changes to the position of the head end 46 of the support apparatus 12, time of a recent call to the caregiver, time since the last call, substance of the calls to the caregiver, call attempts from the pillow speaker (e.g., the handheld user interface 92), types of calls, quantity of calls, frequency of calls, etc.

Additionally, the locator system 146 may also provide information to the monitoring system 10. This information includes, but is not limited to: the location of the assigned caregiver in the medical facility, a distance or time for the assigned caregiver to reach the patient room 40 from the current location, location of additional caregivers, etc. Further, the coding system 150 may also provide information to the monitoring system 10. This information may include, but is not limited to: code status of the unit or floor where the patient is located or rooms in the unit or floor, time since the last code situation for the unit or floor where the patient is located, other units or floors in a current code situation, etc. The monitoring system 10 may monitor single events, patterns of the events over a predefined period of time, trends in the events, or a combination thereof. Depending on the current code of the coding system 150, the monitoring system 10 is configured to increase the sensitivity of the fall risk protocol for one, some, or all patients in the unit, floor, or the medical facility in response to the current code.

With further reference to FIGS. 6 and 12, the monitoring system 10 may also retrieve a variety of information regarding the patient from the EMR 180. The information in the EMR 180 may be input by the caregiver or automatically added or updated by other systems and devices within the medical facility. This information may include, but is not limited to: whether the patient is a repeat fall risk offender (e.g., attempting bed exits without assistance), the patient has repeat false alarms for fall risk (e.g., repeated non-exit movements that trigger an alert), a Morse score, a Hendrich score, a score from the Johns Hopkins Fall Risk Assessment Tool (JHFRAT), a Get Up and Go test score, medication effects, timing of medication doses, attachments to the patient (e.g., IVs, cuffs, etc.), whether the patient uses assistive walking devices, the facility protocol for fall risk, mental status or orientation to own abilities, the history of fall risk, history of prior unassisted exits, frequency or urgency of urination, bibliographic information (e.g., name, room number, etc.), vital signs information (e.g., heart rate, respiration rate, blood pressure, etc.), therapies (e.g., with a pneumatic system), medications, etc.

Several factors from the EMR 180 may be determined by the caregiver at a certain time or times during the stay of the patient at the medical facility. For example, the Morse score may be determined by the caregiver. The Morse score includes multiple factors, including a history of fall risk, secondary diagnosis, ambulatory aids, IVs, gait, and mental status. In another example, the Hendrich score may be determined by the caregiver and may be used in addition to or in lieu of the Morse score. The Hendrich score includes different factors, including recent history, altered elimination, depression, dizziness or vertigo, poor mobility or weakness, confusion or disorientation, and poor judgment. The JHFRAT has a preliminary determination that automatically categorizes a patient as high fall risk per protocol (i.e., history of falls in six months before admission, fall during this hospitalization, or protocol such as seizure precautions) or low fall risk per protocol (paralysis or immobility). If the automatic categorization does not apply to the patient, the JHFRAT uses age; fall history; elimination, bowel, and urine; medications; mobility; and cognition as factors for determining whether a patient is a low, moderate, or high fall risk.

The Get Up and Go Test may also be determined by the caregiver and may be included as part of the Hendrich test. The Get Up and Go Test is indicative of trunk strength, which is associated with static and dynamic balance as well as falls. The Get Up and Go Test measures the time it takes for the patient to stand from the chair, walk a certain distance, turn around, walk to the chair, and again sit on the chair. If the time is above a certain threshold, the patient is at risk of falling. Further, the Get Up and Go Test indicates strength and balance by how the patient elevates from the chair, including rising in a single movement, pushing to rise in one attempt, pushing to rise in multiple attempts, and being unable to rise. These may be entered into the EMR 180 and may be conducted as part of admission procedures or when the status of the patient has changed. The monitoring system 10 may monitor single events, patterns of the events over a predefined period of time, trends in the events, or a combination thereof.

Additionally or alternatively, certain therapies assigned to the patient and performed by the support apparatus 12 may also affect the risk evaluation determined by the monitoring system 10. For example, patients who are at risk of developing pressure ulcers may be assigned lateral rotation therapies, which use the pneumatic system in a support surface assembly or mattress for turning the patient between the right and left sides of the body, or alternating low pressure therapies. The movement from the therapy generally adjusts the position of the patient, which is sensed by the patient position sensors 14 and ultimately communicated to the monitoring system 10. When these types of therapies are activated, the sensitivity of the fall risk protocol may be reduced to allow this movement without generating false fall risk alarms.

The position of the support apparatus 12 may also affect the risk evaluation. The bed sensors 70 are configured to sense different positions of the segments 46, 48, 50 of the support apparatus 12. The change in position of the segments 46, 48, 50 may affect the position of the patient relative to the support apparatus 12. For example, an elevated head end 46 may cause the center of gravity of the patient to shift toward the foot end 50. The monitoring area 18 and/or monitoring level 22 of the fall risk protocol may be adjusted based on the position of the support apparatus 12.

With reference still to FIGS. 6 and 12, additional inputs 270 may be utilized by the monitoring system 10 to adjust the overall risk evaluation. Such information may include information from medical or treatment devices (e.g., heart rate monitors, blood pressure cuffs, etc.), additional sensors which may be worn by the patient (e.g., accelerometer, etc.), etc. These devices and sensors may communicate information to the EMR 180 and/or the monitoring system 10. Additional inputs 270 relevant to the patient, the caregiver, or the medical facility may be utilized by the monitoring system 10 without departing from the teachings herein.

The monitoring system 10 collects the inputs, data, and information from the various sources as generally described herein. The monitoring system 10 may weigh the various inputs, compile the inputs, and determine the overall risk evaluation using at least one algorithm or routine 164. The monitoring system 10 is dynamic and adaptive, continually and in real-time receiving new or updated information from the various sources and updating the overall risk evaluation. The status of the patient may change during the stay at the medical facility, including health and cognition, as well as change in the daily schedule as day becomes night. The monitoring system 10 is configured to adapt to these changes.

With each calculation and update of the overall risk evaluation, the sensitivity of the fall risk protocol is adjusted or updated. The sensitivity of the fall risk protocol generally refers to when an alert is triggered relative to the movement of the patient. The higher the sensitivity, the earlier the alert is triggered based on the position of the patient. Accordingly, the lower the sensitivity, the later in the exit process the alert is triggered.

The sensitivity of the fall risk protocol is adjusted by adjusting the monitoring level 22 for the monitoring area 18. For example, the distance from predefined locations may be adjusted. In such examples, the monitoring area 18 within a shorter distance to the predefined locations, lowers the monitoring level 22 of the monitoring system 10. The monitoring area 18 within a greater distance from the predefined locations, increases the monitoring level 22.

In additional non-limiting examples, the sensitivity or monitoring level 22 is adjusted by adjusting the boundary 24 and, consequently, the size or geometry of the monitoring area 18 on the support apparatus 12. The change in the monitoring area 18 may also change as a percentage relative to a baseline or an initial monitoring area 18 determination. The boundary 24 may be moved closer to an edge of the support apparatus 12 to increase the size of the monitoring area 18 and decrease the sensitivity. Accordingly, movement near or outside the boundary 24 is a movement closer to an exit attempt. The boundary 24 may be moved closer to a center of the support apparatus 12 to decrease the size of the monitoring area 18 and increase the sensitivity. Accordingly, movement near or outside the boundary 24 is a more initial exit movement, further in time from the exit attempt. The change in the boundary 24 may form concentric shapes of the monitoring area 18, similar to the concentric trapezoids illustrated in FIGS. 4 and 12. Additionally or alternatively, the shape and the size of the monitoring area 18 may change. In such examples, a head end of the monitoring area 18 may change separately from a base or foot end of the monitoring area 18, which may be advantageous to provide more customized monitoring areas 18 based on the patient.

Referring again to FIGS. 6 and 12, the overall risk evaluation and corresponding sensitivity or monitoring level 22 may be any value on a predefined scale. In non-limiting examples, the overall fall risk evaluation may be a number between zero and 100. The predefined scale may have n-number of values, creating a more proportional and customizable risk evaluation based on numerous factors. With n-number of values, the monitoring area 18 may have an n-number of strata depending on the monitoring system 10, creating a more proportional change to the sensitivity. Each risk score evaluation adjusts the monitoring level 22 and, therefore, the sensitivity of the fall risk protocol.

The support apparatus 12 and the monitoring system 10 utilize the monitoring level 22 of the monitoring trigger (e.g., the boundary 24, the movement, the distance to the predefined position, etc.) to determine whether the patient is attempting to exit the bed when under the fall risk protocol. The patient position sensor 14 of the support apparatus 12 monitors the position or movement of the patient on the support apparatus 12. In certain aspects, the patient position is communicated directly to the monitoring system 10 to be compared to the monitoring area 18. Alternatively, the control unit 16 of the support apparatus 12 compares the position of the patient to the monitoring area 18, and the control unit 16 may communicate the comparison to the monitoring system 10.

When the patient position is within the monitoring area 18, the monitoring system 10 may continue to monitor the position or movement and adjust the monitoring level 22 (e.g., to subsequent monitoring levels 22) based on the various inputs. For example, the monitoring system 10 is configured to adjust the distance, threshold, or boundary 24 to subsequent distances, thresholds, or boundaries 24, respectively.

For example, in configurations where the monitoring level 22 is implemented as the adjustable boundary 24, when the patient position crosses the boundary 24 and/or is at least partially outside the boundary 24, a bed exit alert is triggered by the monitoring system 10. The boundary 24 may be dynamically adjusted over time as the factors or inputs to the monitoring system 10 change. Any change to the patient, the caregiver, the support apparatus 12, etc. may affect the sensitivity of the fall risk protocol, which consequently adjusts the boundary 24 of the monitoring area 18.

The monitoring level 22 is adjusted to affect when the bed exit alert is triggered. For example, the lower the sensitivity, the later the alert is triggered based on the patient position. In such examples, there is generally a shorter time between the alert and the exit attempt by the patient. On the other hand, when the monitoring system 10 is at an increased monitoring level 22, the center of gravity of the patient crosses the boundary 24 to be outside of the monitoring system 10 at an earlier stage of a process of exiting the support apparatus 12. The monitoring system 10 will alert the caregiver earlier in the exiting process, allowing more time for the caregiver to reach the patient, increasing the likelihood the caregiver can intervene and prevent the fall hazard.

With further reference to FIGS. 6 and 12, the change in the monitoring level 22 of the fall risk protocol adjusts the time from the initial indication that the patient is attempting to exit the bed (e.g., the patient position is outside of the monitoring area 18) and an estimated time for the caregiver to arrive at the patient room 40 to assist the patient. For example, if the caregiver location is further away from the patient room 40, the sensitivity may be increased to provide more time for the caregiver to reach the patient room 40. In another example, if the unit where the patient room 40 is located is under a current code status, the sensitivity may be increased because the caregivers may be responding to the code, and caregivers from another unit may travel to reach the patient under the fall risk protocol.

The monitoring system 10 may have a temporal aspect to reduce the risk of false alarms, which may lead to alarm fatigue for the caregivers. The temporal aspect may determine whether movement across or outside the boundary 24, movement toward the predefined location, movement over a threshold, etc. is above or below a predetermined time threshold. The time threshold may be a static value or may be dynamically adjusted by the monitoring system 10 in response to patterns, trends, movements, and other factors derived from the information from various information sources.

For example, if the patient moves outside the boundary 24 and back inside the boundary 24 of the monitoring area 18 in a time that is below the time threshold, the fall risk alert may not be generated by the monitoring system 10. These movements may be non-exit movements such as, reaching over the siderail 72 to a side table or adjusting for comfort and may not indicate a bed exit. This may be advantageous for reducing false alarms. If the movement across or outside the boundary 24 is equal to or exceeds the time threshold, the fall risk alert may be generated, as described further herein.

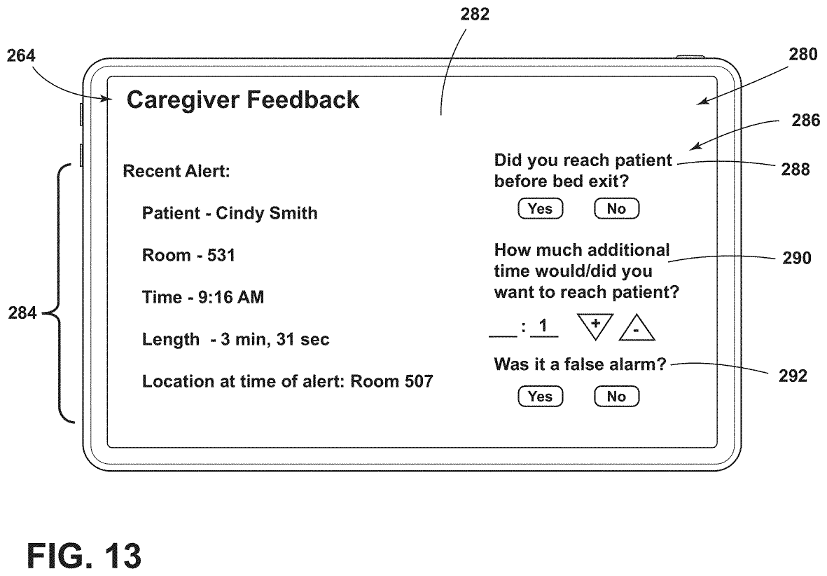

With reference now to FIGS. 12 and 13, the caregiver feedback 264 may be utilized as an input that is factored into the overall risk evaluation and as a method for optimizing the routines 164 that determine the overall risk evaluations. The caregiver feedback 264 may provide an input related to how accurate the adaptive sensitivity of the fall risk protocol is, providing a balance between the sensitivity or monitoring level 22 and the specificity of the monitoring system 10. The feedback 264 is factored into the fall risk evaluation determination and adjustments are made to the evaluation (e.g., the routines 164 that determine the evaluation) and, consequently, the monitoring level 22 in accordance with the feedback 264 and patterns or trends in the feedback 264. The monitoring system 10 may utilize overall trends from all patients and caregivers within the unit, the floor, or the medical facility, as well as individual trends from select patients and caregivers.

The feedback 264 may be entered through an application interface 280, an example of which is illustrated in FIG. 13. The application interface 280 is included in the station device 222 (FIG. 9), the room device 232 (FIG. 1), the remote user device 142 (FIG. 6), etc. Additionally, the application interface 280 is generally a touch screen for viewing and inserting information. After receiving the alert from the monitoring system 10 and attending to the patient, the caregiver may provide the feedback 264 about the alert.

A feedback display 282 on the application interface 280 includes information about the alert. In the illustrated example, alert information 284 includes a patient name, a room number, a time of the alert, a time until the caregiver was determined to be in the patient room 40 (e.g., as determined by the imaging system 130 or the locator system 146), and a location of the caregiver at the time the alert was triggered. Additional data and metrics may also be displayed on the feedback display 282 without departing from the teachings herein.

Referring still to FIGS. 12 and 13, the feedback display 282 also includes response features 286 for the caregiver to provide feedback 264 regarding the accuracy and timing of the monitoring system 10. For example, as illustrated in FIG. 13, the response features 286 may include a timing status 288 where the caregiver may indicate whether the caregiver received the risk fall alert and arrived in the patient room 40 prior to the patient exiting the support apparatus 12. In another example, the response features 286 may include an adjustment indicator 290 where the caregiver can indicate how much additional time would have been helpful between the fall risk alert and the time the caregiver arrived in the patient room 40. Using the adjustment indicator 290, the caregiver can also indicate if less time could have been given to reach the patient room 40. Further, the response features 286 may include a false alarm indicator 292, allowing the caregiver to note whether the fall risk alert was sent and the patient did not attempt the bed exit. Additional information will be input by the caregiver and this information may be communicated to the monitoring system 10 to optimize the routines 164 of the monitoring system 10 that adjusts the overall risk evaluation and corresponding protocol sensitivity.

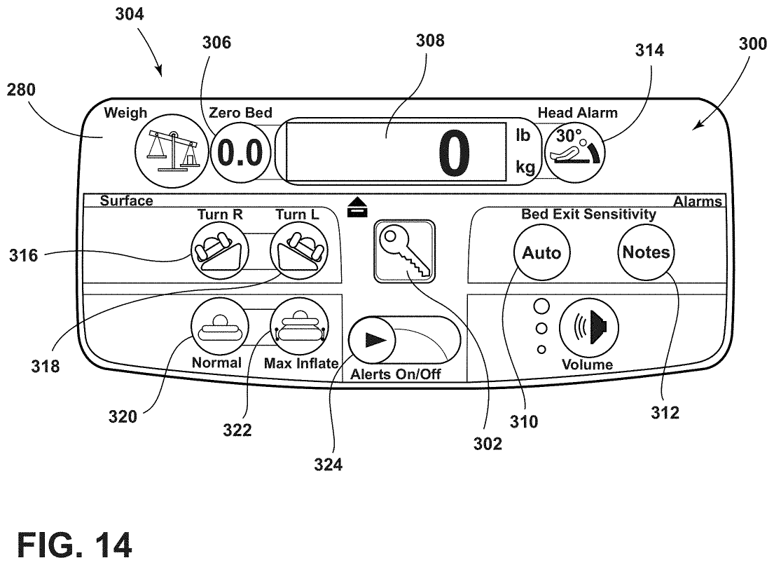

With reference to FIG. 14, the application interface 280 is illustrated as having a bed adjustment display 300. The bed adjustment display 300 includes a variety of selectable features for adjusting or utilizing different features of the support apparatus 12. Each of these features may be locked or unlocked through a key feature 302. The key feature 302 may include a passcode, key fob, or other access granting parameter designed to limit access to the bed adjustment display 300. When the key feature 302 is accessed, the caregiver may input or adjust the information on the application interface 280. The key feature 302 may prevent inadvertent adjustments or adjustments by the patient to the support apparatus 12. In various aspects, the bed adjustment display 300 may include a scale feature 304. The support apparatus 12 utilizes the patient position sensors 14 or other weight sensors to measure a weight of the patient positioned on the support apparatus 12. The scale feature 304 includes a zeroing button 306 for zeroing the scale and a weight status 308 for displaying the weight of the patient.

The bed adjustment display 300 in the illustrated example of FIG. 14 generally includes features directed to at least three categories, including falls, pulmonary, and skin. For fall-related aspects, the application interface 280 is utilized for caregivers to monitor bed position and bed exit alarm status for patients at risk of falls. The caregiver may activate the monitoring system 10 by selecting a sensitivity feature 310 on the application interface 280. The sensitivity feature 310 is configured to activate the monitoring system 10 to automatically adjust the sensitivity of the fall risk protocol as described herein. Further, the application interface 280 may include a feedback feature 312 that adjusts the application interface 280 to the feedback display 282 (FIG. 13), allowing the caregiver to provide information regarding a recent fall risk alert.

For pulmonary-related aspects, the application interface 280 is used for caregivers to monitor head-of-bed angles for patients at risk of pulmonary complications when the head-of-bed angle is outside of a predetermined angle range. The predetermined angle range may be, for example, between about 30° and about 45° relative to a flat position. The bed adjustment display 300 may include a head-of-bed feature 314, which monitors the position or angle of the head end 46 of the support apparatus 12 (e.g., with the bed sensors 70). The head end 46 may be positioned at certain angles or in ranges of angles based on treatments or therapies for the patient. When activated, a bed alarm may alert the caregiver when the head end 46 moves outside the selected position range or when the head end 46 is moving or has reached an angle range limit. It is also contemplated that the caregiver can set limits to the available range to which the head end 46 can be adjusted.

For skin-related aspects, the application interface 280 is used for caregivers to monitor turn reminders for patients at risk of pressure ulcers to automatically remind the caregivers to turn the patient at predefined intervals. The support apparatus 12 may include the pneumatic system within the mattress, which includes bladders that are independently and selectively adjustable between a deployed state and a non-deployed state. In the illustrated example of FIG. 14, the application interface 280 includes turn features 316, 318, which activate the pneumatic system to turn to the patient. This may be advantageous for various skin therapies, such as lateral rotation therapy.

Further, the application interface 280 includes inflation features 320, 322, which adjusts an inflation of the bladders to consequently adjust the thickness of the mattress. Alternatively, the application interface 280 may be configured to allow automatic adjustment of the mattress at predetermined intervals. Accordingly, the caregiver could also select an automated adjustment of the mattress at a selected interval to minimize the risk of the patient of developing a dangerous or uncomfortable skin condition. Further, the caregiver may be notified through the application interface 280 when the patient is being adjusted at each or selected intervals. The notification may also allow the caregiver to assist the patient during the adjustment process. Additionally, the application interface 280 may notify the caregiver when the patient is to be adjusted and the caregiver may manually adjust the patient on the mattress. This notification may also alert the caregiver that the monitoring system 10 may be adjusting the monitoring level 22 in response to the activated therapy.

Referring still to FIG. 14, the application interface 280 may also be used to activate or deactivate alerts with an alert adjustment feature 324. The caregiver may manually adjust whether the application interface 280 provides an alert. Additionally, if an alert is automatically triggered, the caregiver may utilize the application interface 280 to deactivate the alert. For example, if the fall risk alert is triggered the caregiver may deactivate the alert manually via the application interface 280.

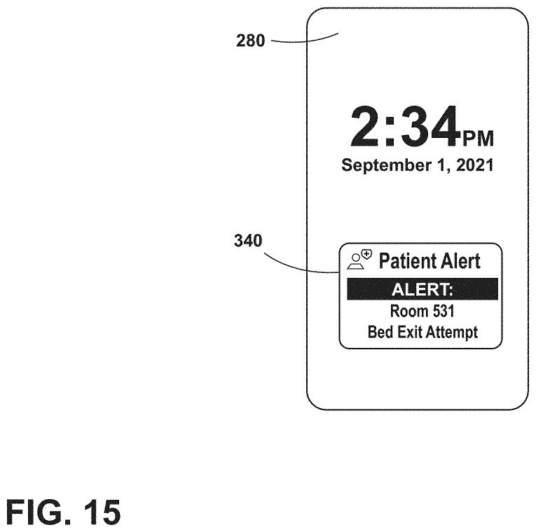

With reference now to FIG. 15, the application interface 280 of the remote user device 142 is illustrated with an exemplary fall risk alert notification 340. When the patient position is sensed at least partially outside the monitoring area 18, the monitoring system 10 generates the fall risk alert notification 340 and communicates the fall risk alert notification 340 to the caregiver, such as in the illustrated push notification. The fall risk alert notification 340 may be displayed on any one or more of the application interfaces 280 associated with the monitoring system 10, including the station device 222, the status board 230, the room device 232, the remote user device 142, etc. The fall risk alert notification 340 indicates to the caregiver that the patient is attempting to exit the support apparatus 12 unassisted while the fall risk protocol is activated and that the caregiver should go to the patient room 40 to assist the patient.

With reference to FIGS. 1-15, when the patient is admitted into the medical facility, the caregiver may assign preliminary or initial fall risk information to the patient. This risk information can include the Morse score, the Hendrich score, the JHFRAT score, the Get Up and Go Test score, or other fall risk information. The fall risk score is entered into the EMR 180, the application interface 280, or a combination thereof as an initial fall risk determination. When the fall risk score is entered directly into the EMR 180, the score will also be communicated to any software or application that utilizes the information, such as the call system 148, the monitoring system 10, etc. The monitoring system 10, the call system 148, or the caregiver may utilize the initial or preliminary fall risk score to assign the patient a fall risk status, which determines whether the fall risk protocol is activated.

If the fall risk protocol is activated, the fall risk alert notification 340 is communicated to the caregiver to inform the caregiver that his or her patient is actively under the fall risk protocol. Further, the status board 230 at the nurse station 220 may display an indicator 342 of the assigned fall risk protocol. Additionally, an indicator light 344 outside of the patient room 40 may be activated to indicate the fall risk status. The indicator light 344 may be activated when the fall risk protocol is initiated and deactivated when the patient is not within the fall risk protocol. Additionally or alternatively, the indicator light 344 may be adjusted by the monitoring system 10 to indicate the sensitivity or monitoring level 22 of the fall risk protocol for that patient. For example, the indicator light 344 may illuminate a first color for lower sensitivity and a second color for higher sensitivity. Alternatively, a top portion of the indicator light 344 may illuminate for higher sensitivity and a lower portion of the indicator light 344 may illuminate for lower sensitivity. The medical facility may have select protocols for indicating the fall risk status of the patient. Higher and lower sensitivities may be displayed as different colors, different intensities of colors, etc.

Referring still to FIGS. 1-15, the monitoring system 10 is in an active state when the fall risk protocol is activated. The monitoring system 10 begins to retrieve information and determine the monitoring level 22, such as the boundary 24, of the monitoring area 18. The monitoring system 10 begins monitoring the patient position for generating the fall risk alert notification 340 when the patient is detected on the support apparatus 12 and the caregiver is detected as exiting the patient room 40. The patient may be detected on the support apparatus 12 by the patient position sensors 14. The caregiver leaving the patient room 40 may be detected by the imaging system 130 and/or the locator system 146 and communicated to the monitoring system 10. Additionally, when the monitoring system 10 is in the active state, a bed indicator 346 may be activated, such as a bed indicator light, to provide a visual indication in the patient room 40 that the monitoring system 10 is activated.

When the monitoring system 10 is activated, the monitoring system 10 is configured to obtain or receive the data and information from the various information sources to determine the overall risk evaluation. In certain aspects, the initial monitoring level 22 may be assigned using the initial risk assessment from the caregiver. The monitoring system 10 may also utilize the preliminary risk evaluation from the control unit 16 to assign the initial monitoring level 22. With each update or additional information from the information sources, the monitoring system 10 is configured to update the overall risk evaluation. With each overall risk evaluation, the monitoring system 10 continuously updates the monitoring level 22 (e.g., the boundary 24, the movement threshold, the distance to the predefined location, etc.) of the monitoring area 18 to adjust the sensitivity of the fall risk protocol.

Referring still to FIGS. 1-15, when the patient position moves outside or above the monitoring level 22 of the monitoring area 18, the monitoring system 10 generates and communicates the fall risk alert notification 340 directly to the caregiver via the remote user device 142. The fall risk alert notification 340 may be generated when the patient position movement is at least partially outside the boundary 24, when the patient position moves within a predefined distance from the predefined location, when the patient movement is above the movement threshold, etc.

In addition to the remote user device 142, the alert notification 340 may also be communicated by the support apparatus 12, the room device 232, the station device 222, the status board 230, etc. In certain aspects, the indicator light 344 outside the room may also be utilized to indicate the patient has moved outside the monitoring area 18. Each alert notification 340 may be similar or may be different depending on which device is communicating the alert notification 340. The alert notification 340 may be visual, haptic, audible, or a combination thereof.

The alert notification 340 is configured to be canceled, deactivated, or suppressed in response to the caregiver entering the patient room 40. The caregiver may be detected by the imaging system 130, the locator system 146, or a combination thereof. Further, the status board 230 may display that the caregiver is currently in the patient room 40, indicating to other members of the caregiving team that the assigned caregiver has responded to the fall risk alert notification 340.

When the patient returns to the support apparatus 12 and the caregiver again exits the patient room 40, the monitoring system 10 automatically adjusts to allow a new fall risk alert notification 340 to be sent upon the next triggering exit-based movement. Additionally, the caregiver provides feedback 264 about the fall risk alert notification 340, which is communicated to the monitoring system 10 to optimize the routines 164.

Additionally, it is contemplated that the monitoring system 10 may be in a “standby” state when the patient leaves the patient room 40, such as for a procedure. In the “standby” state, the monitoring system 10 may continue to obtain information from the various sources and adjust the monitoring level 22, but the monitoring system 10 may not generate the fall risk alert notification 340. Adjustment of the monitoring level 22 while the patient is out of the room allows the monitoring system to exit the “standby” state with the most recent and accurate information about the fall risk status of the patient. The caregiver may enable, set, or input an “out of room” status for the patient. Additionally or alternatively, the “out of room status” may be automatically set in response to information captured in the image data of the imaging system 130. When the patient returns to the patient room 40 and the support apparatus 12, the monitoring system 10 will again be configured to communicate the fall risk alert notification 340 under the select conditions. The re-entry into the patient room 40 may be input by the caregiver, detected by the imaging system 130, and/or detected by the support apparatus 12.

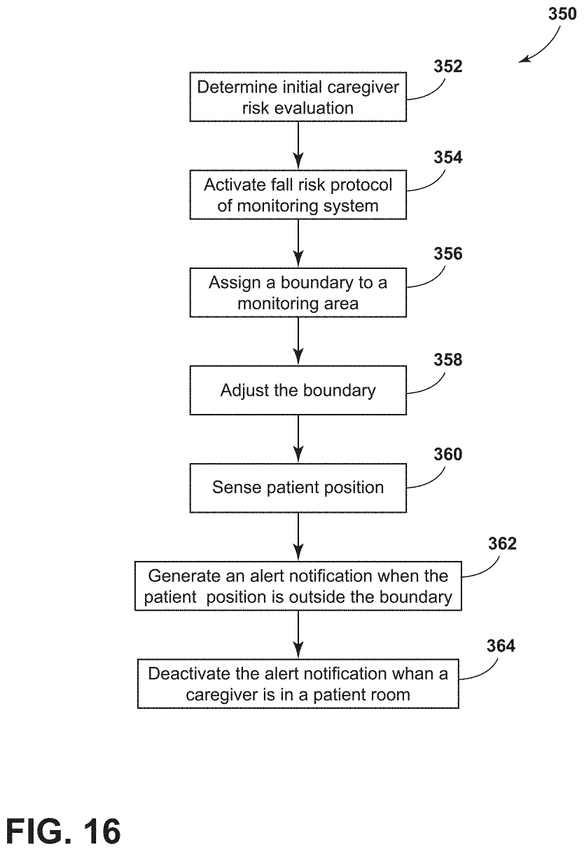

Referring to FIG. 16, as well as to FIGS. 1-15, a method 350 of monitoring the patient under fall risk protocol includes step 352 of determining an initial fall risk evaluation, generally determined by the caregiver. The caregiver evaluates the patient upon admittance to the medical facility or a change in patient status. The caregiver may input the initial fall risk information into the EMR 180. The initial fall risk evaluation is obtained by the monitoring system 10. In step 354, the fall risk protocol is activated, and consequently, the monitoring system 10 is activated. In step 356, the monitoring system 10 assigns the monitoring level 22 of the monitoring area 18 based on initial information. The initial information may be or include the information from the EMR 180, as well as any other information available from the information sources at the time the fall risk protocol is activated.

In step 358, the monitoring system 10 dynamically adjusts the monitoring level 22, such as the distance to the predefined location, the movement threshold, and/or the boundary 24. Further, in step 358, the monitoring system 10 retrieves or receives information and continuously determines the overall fall risk evaluation. In response to the update or re-calculation of the overall risk evaluation, the monitoring system 10 adjusts the monitoring level 22 to adjust the sensitivity of the fall risk protocol. The monitoring system 10 is configured to dynamically adjust the monitoring area 18 based on the continuously changing overall fall risk evaluation.

In step 360, the patient position and movement are sensed and monitored by at least one of the control unit 16 of the support apparatus 12 and the controller 20 of the monitoring system 10. When monitored by the control unit 16, the patient position is communicated to the monitoring system 10 by the support apparatus 12. Further, in examples that include a temporal aspect, the patient position is communicated to the monitoring system 10 when the patient position is outside of the monitoring area 18 or movement above the threshold fora time that is greater than or equal to the time threshold.

In step 362, the monitoring system 10 generates and communicates the fall risk alert notification 340 when the patient position, which generally corresponds with the center of gravity of the patient, crosses or is at least partially outside of the monitoring area 18, within a predefined distance of the predefined location, or above the movement threshold. The fall risk alert notification 340 indicates that the caregiver is to assist the patient in attempting to exit the bed. The timing for the fall risk alert notification 340 depends on the sensitivity of the fall risk protocol, which is dynamically adjusted to allow the caregiver time to reach the patient while reducing false alarms. In step 364, the alert notification 340 is deactivated when the caregiver enters the patient room 40. When the caregiver again exits the patient room 40, a new fall risk alert notification 340 will be generated in response to the movement of the patient outside the boundary 24. It is understood that the steps of the method 350 may be performed in any order, simultaneously, omitted, and/or repeated without departing from the teachings provided herein.

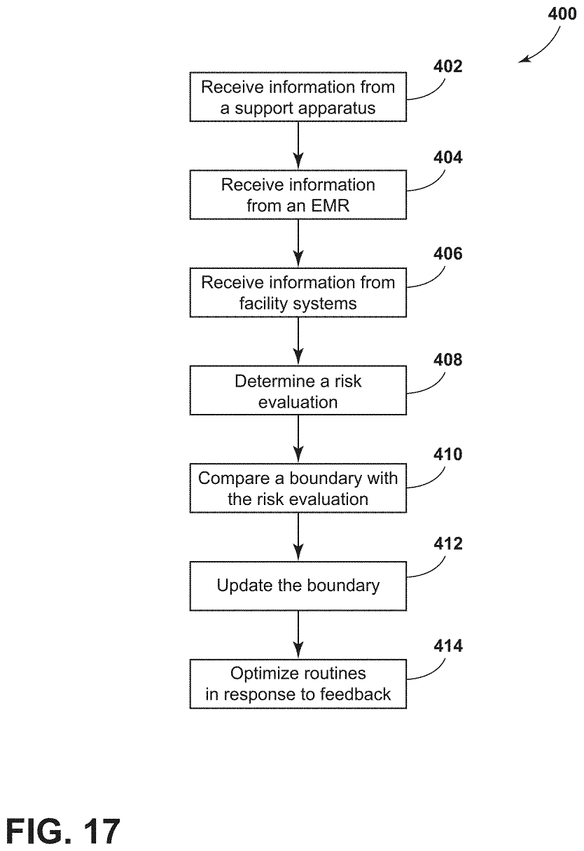

With reference now to FIG. 17, as well as FIGS. 1-16, a method 400 of adjusting the sensitivity of the fall risk protocol may be utilized to dynamically adjust the monitoring area 18. The method 400 may be separate from or part of the method 350. The method 400 includes step 402 where the monitoring system 10 receives the information and data from the support apparatus 12. This information and data may include sensed information, as well as the preliminary risk evaluation determined by the control unit 16. In step 404, the monitoring system 10 receives the information and data from the EMR 180 for the patient. In step 406, the monitoring system 10 receives the information and data from various facility systems, including, but not limited to, the call system 148, the imaging system 130, the coding system 150, and the locator system 146.

In step 408, the monitoring system 10 determines the overall risk evaluation in response to the received information. The overall risk evaluation is calculated using at least one of the routines 164, which may compile, weigh, sort, classify, or otherwise utilize the information to make a calculation. In step 410, the controller 20 may compare the current monitoring level 22, such as the current boundary 24, distance to the predefined location, or the movement threshold of the monitoring system 10 with the calculated fall risk evaluation.

The current monitoring level 22 may be an initial monitoring level 22 (e.g., boundary 24, distance, or threshold) based on initial information upon activation of the fall risk protocol or a previous monitoring level 22 that was determined based on a previous overall risk evaluation. In step 412, the monitoring system 10 may update the monitoring level 22 in response to the newest or most up-to-date overall risk evaluation. The monitoring system 10 may continuously receive information, update the overall risk score, and dynamically adjust the monitoring level 22, consequently adjusting the distance to the predefined location, the movement threshold, and/or the boundary 24 of the monitoring area 18.

In step 414, the caregiver feedback 264 is communicated to the monitoring system 10. The monitoring system 10 utilizes the feedback 264 to optimize the routines 164 (e.g., fall risk routines 164) that calculate the overall risk evaluation to improve the accuracy of the routines 164. The monitoring system 10 factors the caregiver feedback 264 into the adjustment of the fall risk protocol sensitivity. The monitoring system 10 may adjust the routines 164 in response to the feedback 264. Consequently, by updating the routines 164, the monitoring system 10 may ultimately adjust the boundary 24 in response to the caregiver feedback 264. It is understood that the steps of the method 400 may be performed in any order, simultaneously, omitted, and/or repeated without departing from the teachings provided herein.

Referring to FIGS. 1-17, the monitoring system 10 provides an adaptive system for the fall risk protocol that factors a variety of patient, caregiver, and facility information to adjust the sensitivity of the protocol. The adjustment in the sensitivity changes the time the caregiver is given to respond to the fall risk alert notification 340. With an increased sensitivity, initial movement indicative of a bed exit causes the patient position to be at least partially outside of the smaller monitoring area 18 and results in an alert to the caregiver, allowing the caregiver more time to reach the patient before the bed exit attempt. With a lowered sensitivity, later or subsequent movement indicative of the bed exit causes the patient position to be at least partially outside the monitoring area 18 and results in the alert to the caregiver, allowing a lesser amount of time to reach the patient before the bed exit attempt. The amount of time determined by the monitoring system 10 generally provides a sufficient amount of time for the caregiver to reach the patient and prevent the potential fall hazard based on the information weighed by the monitoring system 10.

Use of the present system may provide for a variety of advantages. For example, the monitoring system 10 may utilize a variety of information to adjust the monitoring area 18 for the fall risk protocol. Further, the monitoring system 10 may utilize information about the patient, the caregiver, and the facility to adjust the system sensitivity. Moreover, the monitoring system 10 is configured to adapt based on where the caregiver is located and what the caregiver is doing, rather than assume a same or standard response time to each alert. Also, the monitoring system 10 may utilize the caregiver feedback 264 to optimize the routines used to dynamically adjust the monitoring system 10. Additionally, the monitoring system 10 may generate the fall risk alert notification 340 based on the overall risk evaluation, which generally provides sufficient time for the caregiver to reach the patient prior to the bed exit. Further, the monitoring system 10 is adaptive and removes the fall risk sensitivity determination from the caregiver. Moreover, the adaptive monitoring system 10 may have n-number of monitoring areas 18 or monitoring levels 22, providing a system that is more proportional and more customized to the patient, the caregiver, and the facility conditions. Additional benefits or advantages may be realized and/or achieved.

Each of the controllers and the control units disclosed herein may include various types of control circuitry, digital or analog, and may each include a processor, a microcontroller, an application specific integrated circuit (ASIC), or other circuitry configured to perform the various inputs or outputs, control, analysis, or other functions described herein. The memories described herein may be implemented in a variety of volatile and nonvolatile memory formats. Routines may include operating instructions to enable the various methods described herein.

Related applications, for example those listed herein, are fully incorporated by reference. Descriptions within the related applications are intended to contribute to the description of the information disclosed herein as may be relied upon by a person of ordinary skill in the art. Any changes between any of the related applications and the present disclosure are not intended to limit the description of the information disclosed herein, including the claims. Accordingly, the present application includes the description of the information disclosed herein as well as the description of the information in any or all of the related applications.

The system disclosed herein is further summarized in the following paragraphs and is further characterized by combinations of any and all of the various aspects described therein.

According to another aspect of the present disclosure, a monitoring system for a patient fall risk protocol includes a support apparatus that includes a sensor configured to sense a position of a patient. A control unit is in communication with the sensor. The control unit is configured to monitor the position of the patient relative to a monitoring area on the support apparatus. The control unit is configured to determine a preliminary fall risk evaluation. A controller is in communication with the support apparatus. The controller is configured to receive the preliminary fall risk evaluation from the support apparatus, determine an overall fall risk evaluation using the preliminary fall risk evaluation, assign a monitoring level for the monitoring area based on the overall fall risk evaluation, adjust the overall fall risk evaluation, and adjust the monitoring level of the monitoring area in response to changes in the overall fall risk evaluation.

According to another aspect of the present disclosure, a control unit is configured to communicate a position of a patient to a controller when the position is at least partially outside a boundary of a monitoring area. A controller is configured to generate an alert notification in response to the position of the patient being at least partially outside the boundary of the monitoring area.

According to another aspect of the present disclosure, a controller is configured to adjust a monitoring level to decrease a size of a monitoring area within a boundary and increase a sensitivity for a fall risk routine of the controller to adjust when the controller is configured to generate an alert notification.

According to another aspect of the present disclosure, a controller is configured to adjust a monitoring level to increase a size of a monitoring area within a boundary and decrease a sensitivity for a fall risk routine of the controller to adjust when the controller is configured to generate an alert notification.

According to another aspect of the present disclosure, a controller is configured to generate an alert notification when a position of the patient is sensed at least partially outside of a boundary.

According to another aspect of the present disclosure, a controller is configured to communicate an alert notification to an application interface.

According to another aspect of the present disclosure, an application interface is included in at least one of a remote user device and a device of a call system.

According to another aspect of the present disclosure, a device of a call system includes at least one of a station device at a nurse station, a status board, and a room device in a patient room.

According to another aspect of the present disclosure, a controller is configured to receive a caregiver location from a caregiver locator system and adjust an overall fall risk evaluation in response to a caregiver location.

According to another aspect of the present disclosure, a controller is configured to retrieve patient information from an electronic medical record and adjust an overall fall risk evaluation in response to patient information. The patient information includes at least one of a fall risk assessment, vital signs information, and medication information.

According to another aspect of the present disclosure, patient information includes at least one of a fall risk evaluation, vital signs information, and medication information.

According to another aspect of the present disclosure, an imaging system has an imager defining a field of view that includes a support apparatus. A controller is configured to monitor movement of a patient in image data received from the imaging system and adjust an overall fall risk evaluation in response to the movement.

According to another aspect of the present disclosure, an imager is at least one of a camera, a three-dimensional image sensor, and an infrared image sensor.

According to another aspect of the present disclosure, a field of view includes an area adjacent to a support apparatus to monitor ambulation of a patient.

According to another aspect of the present disclosure, a support apparatus includes bed sensors configured to sense at least one of a position of segments of the support apparatus, a height of the support apparatus relative to an underlying floor surface, and a siderail position. A controller is configured to adjust an overall fall risk evaluation in response to sensed information from the bed sensors.

According to another aspect of the present disclosure, a fall risk monitoring system for a medical facility includes a support apparatus including a sensor configured to sense position information related to a patient on the support apparatus and a control unit configured to monitor sensed patient information from the sensor relative to a monitoring trigger. A locator system is configured to determine a location of a caregiver in said medical facility. A controller is in communication with the support apparatus and the locator system. The controller is configured to assign a monitoring level of the monitoring trigger based on a fall risk evaluation, receive data from the support apparatus related to the patient and the support apparatus, receive the location of the caregiver, and adjust the monitoring level of the monitoring trigger in response to at least one of the data from the support apparatus and the location of the caregiver.

According to another aspect of the present disclosure, a monitoring trigger is a boundary around a monitoring area on a support apparatus. A sensor is configured to sense a center of gravity of a patient.

According to another aspect of the present disclosure, a control unit is configured to monitor a center of gravity relative to a boundary. A controller is configured to generate an alert notification when the center of gravity is at least partially outside the boundary of a monitoring area.

According to another aspect of the present disclosure, a monitoring trigger is a movement threshold and a sensor is configured to sense movement of a patient on a support apparatus. A controller is configured to generate an alert notification when sensed movement of the patient exceeds the movement threshold.

According to another aspect of the present disclosure, a monitoring trigger is an area within a predefined distance from a side of the support apparatus, wherein the sensor is configured to sense a position of the patient and the control unit is configured to monitor the position of the patient relative to the area within the predefined distance from the side of the support apparatus, and wherein the controller is configured to generate an alert notification when the sensed position of the patient is sensed within the area.

According to another aspect of the present disclosure, data includes patient information and bed information.

According to another aspect of the present disclosure, patient information includes at least one of movement of a patient, bed exit attempts, and a center of gravity of the patient.

According to another aspect of the present disclosure, a support apparatus includes bed sensors configured to sense bed information. The bed information includes at least one head end position, bed height, and siderail positions. A controller is configured to adjust a monitoring level of a monitoring trigger in response to bed information.

According to another aspect of the present disclosure, a call system is in communication with a controller. The controller receives call information and adjusts the monitoring level in response to the call information.

According to another aspect of the present disclosure, a call system includes a pillow speaker.

According to another aspect of the present disclosure, call information includes at least one of frequency, type, and quantity of calls to a caregiver.

According to another aspect of the present disclosure, an imaging system has an imager defining a field of view that includes a bed. A controller is configured to determine when a caregiver is positioned proximate to a patient in response to image data from the imaging system.

According to another aspect of the present disclosure, a controller is configured to monitor patient movement on a bed based on image data from an imaging system. The controller is configured to adjust a monitoring level in response to the patient movement.

According to another aspect of the present disclosure, a controller is configured to receive facility code information from a coding system and adjust a monitoring level of a monitoring area in response to a current code status.

According to another aspect of the present disclosure, an adaptive patient monitoring system includes a support apparatus having sensors, a servicer storing an electronic medical record, and a controller in communication with the support apparatus and the server. The controller is configured to receive information from the support apparatus relating to at least one of the support apparatus and a person on the support apparatus, receive data from the electronic medical record for the person, determine a monitoring level of a monitoring area on the support apparatus, and adjust the monitoring level in response to at least one of the information from the support apparatus and the data from the electronic medical record.

According to another aspect of the present disclosure, information includes a position of a person on a support apparatus sensed by a sensor in the support apparatus.

According to another aspect of the present disclosure, a controller is configured to generate an alert notification when a position of a person is at least partially outside of a monitoring area.

According to another aspect of the present disclosure, a controller is configured to communicate an alert notification to an application interface.

According to another aspect of the present disclosure, a monitoring level is adjusted an initial monitoring level is an initial boundary of a monitoring area to a subsequent boundary of the monitoring area. The monitoring area in the subsequent boundary is smaller than the monitoring area in an initial boundary to increase a sensitivity of a fall risk protocol to adjust when a controller is configured to generate an alert notification in response to a patient being outside the monitoring area.

According to another aspect of the present disclosure, a controller is configured to communicate with an imaging system configured to capture image data of a support apparatus, monitor patient movement on the support apparatus based on the image data from the imaging system, and adjust a monitoring level in response to the patient movement.

According to another aspect of the present disclosure, a controller is configured to receive facility code information from a coding system and adjust a monitoring level of a monitoring area in response to a current code status.

According to another aspect of the present disclosure, a controller is configured to determine a fall risk evaluation, adjust the fall risk evaluation in response to at least one of information from a support apparatus and data from an electronic medical record, and dynamically adjust a monitoring level in response to the updated fall risk evaluation.

According to another aspect of the present disclosure, a method for monitoring a fall risk patient includes obtaining an initial fall risk evaluation, activating a fall risk protocol of a monitoring system, assigning a monitoring level for a monitoring area on a support apparatus, adjusting the monitoring level of the monitoring area in response to at least one of patient information, caregiver information, and information from the support apparatus, sensing a patient position relative to the monitoring area, and generating an alert notification when the patient position is at least partially outside of the monitoring area.

According to another aspect of the present disclosure, a step of assigning a monitoring level includes assigning the monitoring level based on an initial fall risk evaluation.

According to another aspect of the present disclosure, a step of adjusting a monitoring level includes increasing a size of a monitoring area to decrease a sensitivity of a fall risk protocol.

According to another aspect of the present disclosure, a step of adjusting a monitoring level includes decreasing a size of the monitoring area to increase a sensitivity of a fall risk protocol.

According to another aspect of the present disclosure, a method of adjusting a fall risk protocol includes receiving at least one of sensed patient information and bed information from a bed, receiving stored patient information from an electronic medical record, determining a fall risk evaluation in response to at least one of the sensed patient information, the bed information, and the stored patient information, and adjusting a monitoring level of a monitoring area on the bed in response to the fall risk evaluation.

According to another aspect of the present disclosure, a method includes updating a fall risk evaluation in response to at least one of updated sensed information, updated bed information, and updated stored patient information.

According to another aspect of the present disclosure, a method includes updating a fall risk routine in response to caregiver feedback.

According to another aspect of the present disclosure, an adaptive patient monitoring system includes a support apparatus having a sensor configured to sense a position of a person. The support apparatus includes a control unit that defines a monitoring area on the support apparatus. The control unit is configured to determine a preliminary risk evaluation in response to at least one of movement of the support apparatus and the person. An imaging system defines a field of view that includes the support apparatus. A controller is configured to communicate with the support apparatus and the imaging system. The controller is configured to retrieve a risk score from an electronic medical record, retrieve a caregiver location, determine movement of the person based on image data received from the imaging system, receive the preliminary risk evaluation from the support apparatus, adjust a boundary of the monitoring area in response to at least one of the risk score, the caregiver location, the movement of the person, and the preliminary risk evaluation, and generate an alert when the person moves outside the boundary of the monitoring area.

According to another aspect of the present disclosure, a controller is configured to generate an alert when a person is outside the boundary for a time that is equal to or greater than a predetermined time threshold.

A means for monitoring a patient for a fall risk protocol includes a support means. The support means includes a sensing means configured to sense a position of a patient and an apparatus control means in communication with the sensing means. The apparatus control means is configured to monitor the position of the patient relative to a monitoring area on the support means. The apparatus control means is also configured to determine a preliminary risk evaluation. A system control means is in communication with the support means. The system control means is configured to receive the preliminary risk evaluation from the support means and adjust a boundary of the monitoring area in response to the preliminary risk evaluation.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes, and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A monitoring system for a patient fall risk protocol, comprising:
a support apparatus including a sensor configured to sense a position of a patient and a control unit in communication with the sensor, wherein the control unit is configured to:
monitor the position of the patient relative to a monitoring area on the support apparatus; and
determine a preliminary fall risk evaluation; and
a controller in communication with the support apparatus, wherein the controller is configured to:
receive the preliminary fall risk evaluation from the support apparatus;
determine an overall fall risk evaluation using the preliminary fall risk evaluation;
assign a monitoring level for the monitoring area based on the overall risk evaluation;
adjust the overall risk evaluation; and
adjust the monitoring level of the monitoring area in response to changes in the overall fall risk evaluation.

2. The monitoring system of claim 1, wherein the control unit is configured to communicate the position of the patient to the controller when the position is at least partially outside a boundary of the monitoring area, and wherein the controller is configured to generate an alert notification in response to the position of the patient being at least partially outside of the boundary of the monitoring area.

3. The monitoring system claim 2, wherein the controller is configured to adjust the monitoring level to decrease a size of the monitoring area within the boundary and increase a sensitivity for a fall risk routine of the controller to adjust when the controller is configured to generate the alert notification.

4. The monitoring system of claim 2, wherein the controller is configured to adjust the monitoring level to increase a size of the monitoring area within the boundary and decrease a sensitivity for a fall risk routine of the controller to adjust when the controller is configured to generate the alert notification.

5. The monitoring system of claim 1, wherein the controller is configured to:
receive a caregiver location from a caregiver locator system; and
adjust the overall fall risk evaluation in response to the caregiver location.

6. The monitoring system of claim 1, wherein the controller is configured to:
retrieve patient information from an electronic medical record, wherein the patient information includes at least one of a fall risk assessment, vital signs information, and medication information; and
adjust the overall fall risk evaluation in response to the patient information.

7. The monitoring system of claim 1, further comprising:
an imaging system having an imager defining a field of view that includes the support apparatus, wherein the controller is configured to monitor movement of the patient in image data received from the imaging system and adjust the overall fall risk evaluation in response to the movement.

8. The monitoring system of claim 1, wherein the support apparatus includes bed sensors configured to sense at least one of a position of segments of the support apparatus, a height of the support apparatus relative to an underlying floor surface, and a siderail position, and wherein the controller is configured to adjust the overall fall risk evaluation in response to sensed information from the bed sensors.

9. A fall risk monitoring system for a medical facility, comprising:
a support apparatus including:
a sensor configured to sense patient information related to a patient on the support apparatus; and
a control unit configured to monitor the sensed patient information from the sensor relative to a monitoring trigger;
a locator system configured to determine a location of a caregiver in said medical facility; and
a controller in communication with the support apparatus and the locator system, wherein the controller is configured to:
assign a monitoring level for the monitoring trigger based on a fall risk evaluation;
receive data from the support apparatus related to the patient and the support apparatus;
receive the location of the caregiver; and
adjust the monitoring level of the monitoring trigger in response to at least one of the data from the support apparatus and the location of the caregiver.

10. The fall risk monitoring system of claim 9, wherein the monitoring trigger is a boundary around a monitoring area on the support apparatus, wherein the sensor is configured to sense a center of gravity of the patient.

11. The fall risk monitoring system of claim 10, wherein the control unit is configured to monitor the center of gravity relative to the boundary, and wherein the controller is configured to generate an alert notification when the center of gravity is at least partially outside the boundary of the monitoring area.

12. The fall risk monitoring system of claim 9, wherein the monitoring trigger is a movement threshold and the sensor is configured to sense movement of the patient on the support apparatus, and wherein the controller is configured to generate an alert notification when the sensed movement of the patient exceeds the movement threshold.

13. The fall risk monitoring system of claim 9, wherein the monitoring trigger is an area within a predefined distance from a side of the support apparatus, wherein the sensor is configured to sense a position of the patient and the control unit is configured to monitor the position of the patient relative to the area within the predefined distance from the side of the support apparatus, and wherein the controller is configured to generate an alert notification when the sensed position of the patient is sensed within the area.

14. The fall risk monitoring system of claim 9, wherein the patient information includes at least one of movement of the patient, bed exit attempts, and a center of gravity of the patient.

15. The fall risk monitoring system of claim 9, wherein the support apparatus includes a bed sensor configured to sense bed information, wherein the bed information includes at least one head end position, bed height, and siderail positions, and wherein the controller is configured to adjust the monitoring level of the monitoring trigger in response to the bed information.

16. An adaptive patient monitoring system, comprising:
a support apparatus having sensors;
a server storing an electronic medical record; and
a controller in communication with the support apparatus and the server, wherein the controller is configured to:
receive information from the support apparatus relating to at least one of the support apparatus and a person on the support apparatus;
receive data from the electronic medical record related to the person;
determine a monitoring level of a monitoring area on the support apparatus; and
adjust the monitoring level in response to at least one of the information from the support apparatus and the data from the electronic medical record.

17. The adaptive patient monitoring system claim 16, wherein the monitoring level is adjusted from an initial boundary of the monitoring area to a subsequent boundary of the monitoring area, wherein the monitoring area in the subsequent boundary is smaller than the monitoring area in the initial boundary to increase a sensitivity of a fall risk protocol to adjust when the controller is configured to generate an alert notification in response to a patient being outside the monitoring area.

18. The adaptive patient monitoring system of claim 16, wherein the controller is configured to:
communicate with an imaging system configured to capture image data of the support apparatus;
monitor patient movement on the support apparatus based on the image data from the imaging system; and
adjust the monitoring level in response to the patient movement.

19. The adaptive patient monitoring system of claim 16, wherein the controller is configured to receive facility code information from a coding system and adjust the monitoring level of the monitoring area in response to a current code status.

20. The adaptive patient monitoring system of claim 16, wherein the controller is configured to:
determine a fall risk evaluation;
adjust the fall risk evaluation in response to at least one of the information from the support apparatus and the data from the electronic medical record; and
dynamically adjust the monitoring level in response to the updated fall risk evaluation.
